(19) United States
(12) Patent Application Publication
(10) Pub. No.: US 2024/0000976 A1
(43) Pub. Date: Jan. 4, 2024

(54) ROOM-TEMPERATURE PHOSPHORESCENCE NANOPARTICLES AND METHODS OF MAKING THE SAME

(71) Applicant: THE REGENTS OF THE UNIVERSITY OF MICHIGAN, Ann Arbor, MI (US)

(72) Inventors: Jinsang Kim, Ann Arbor, MI (US); Yingying Zeng, Ann Arbor, MI (US); Do Hyun Kang, Ann Arbor, MI (US); Yannis Paulus, Ann Arbor, MI (US); Van Phuc Nguyen, Ann Arbor, MI (US)

(21) Appl. No.: 18/039,301

(22) PCT Filed: Dec. 3, 2021
(86) PCT No.: PCT/US21/61862

§ 371 Date: May 30, 2023

Related U.S. Application Data
(60) Provisional application No. 63/121,688, filed on Dec. 4, 2020.

(51) Int. Cl. A61K 49/00; C09K 11/02; C09K 11/06; A61P 9/10

(52) U.S. Cl. CPC A61K 49/0015; C09K 11/025; C09K 11/06; A61K 49/0093; A61P 9/10; C09K 2211/1007; C09K 2211/1018

(57) ABSTRACT

Provided herein are room-temperature phosphorescence nanoparticles, methods of preparing the same, and uses of the same.

### CROSS-REFERENCE TO RELATED APPLICATION

The benefit of priority to U.S. Provisional Application No. 63/121,688 filed Dec. 4, 2020 is hereby claimed and the disclosure is incorporated herein by reference in its entirety.

### STATEMENT OF GOVERNMENT SUPPORT

This invention was made with government support under grant number DMR1435965, awarded by the National Science Foundation (NSF) and grant number 1K08EY027458, awarded by the National Institutes of Health. The government has certain rights in the invention.

### BACKGROUND

Ischemia-induced retinal hypoxia is a common complication that can lead to neovascularization, vision impairment, and blindness in several diseases, including proliferative diabetic retinopathy (PDR), sickle cell retinopathy (SCR), retinopathy of prematurity (ROP), and retinal vein occlusion (RVO) (a. Rehak, et al. Current eye research 2008, 33 (2), 111-131; b. Gariano, et al. Nature 2005, 438 (7070), 960-966). RVO is the second most common retinal vascular disorder and represents a major cause of vision loss, affecting more than 16 million people worldwide. In RVO, when the central retinal vein or branch retinal vein is occluded, the retina within the affected vasculature can become ischemic and thereby become hypoxic (Ashton, The British journal of ophthalmology 1954, 38 (7), 385). The development of retinal hypoxia stimulates vascular endothelial cell growth factor (VEGF), hypoxia-inducible factor (HIF), or angiopoietin-like protein-4 to activate angiogenesis, which can lead to vitreous hemorrhage, preretinal fibrovascular membrane formation, tractional retinal detachment, and permanent vision loss in patients (a. Robbins, et al., Growth Factors 1997, 14 (4), 229-241; b. Xin, et al., Proceedings of the National Academy of Sciences 2013, 110 (36), E3425-E3434). RVO is a very heterogeneous disease with highly variable visual acuity outcomes, and it is important that physicians be able to prognosticate outcomes with patients to both counsel them on treatments and set appropriate expectations. Therefore, it is important to detect ischemia-induced hypoxia, the most important pathogenic and prognostic factor of RVO, to better understand RVO pathogenesis and angiogenesis. In addition, long-term visualization and quantification of retinal hypoxia are strongly desirable and will lead to early detection of hypoxia, a better understanding of the pathophysiology of ischemic retinopathies, precise management, and treatment in several diseases including PDR, SCR, ROP, and RVO.

Recently, several techniques have been developed to monitor hypoxia such as oxygen-sensitive microelectrodes, MRI, flow oximetry system, dual wavelength retinal oximetry, and fluorescence and phosphorescence lifetime imaging (a. Feenstra, et al., Early Events in Diabetic Retinopathy and Intervention Strategies 2018, 47; b. Cringle, et al., Investigative ophthalmology & visual science 2002, 43 (6), 1922-1927; c. Berkowitz, et al., Magnetic Resonance in Medicine: An Official Journal of the International Society for Magnetic Resonance in Medicine 2001, 46 (2), 412-416; d. Stefánsson, et al., Progress in Retinal and Eye Research 2019, 70, 1-22). However, these methods each have their limitations. For example, oxygen-sensitive microelectrode is a point measurement technique and thus is highly dependent on the position of the electrode. In tissue with focal hypoxia surrounded by large areas of normoxia, multiple measurements are required, and hypoxic regions can be missed. In addition, this technique is a destructive procedure that requires the implantation of microelectrodes, which thus limits their clinical utility. MRI is a minimally invasive approach, provides a large field of view and depth information. One significant limitation of MRI is that it is not able to provide enough resolution to identify small areas of focal hypoxia. Retinal oximetry is a non-invasive method to measure the percentage of hemoglobin oxygen saturation in retinal vessels based on the difference between optical absorption properties of oxygenated and deoxygenated hemoglobin. This technique offers several advantages such as accurate and reliable measurement of oxygen saturation in retinal vasculature, and is commercially available. However, this technique is restricted to measuring oxygen saturation in large vessels and may not detect the dynamic changes observed in retinal progression such as microaneurysms or capillaries. In addition, it is unable to measure choroidal oximetry, which provides oxygen to the central vision or fovea, and it is unable to measure the oxygen tension outside of larger vasculature within the tissue itself where there can be regions of hypoxia. Fluorescence and phosphorescence-lifetime imaging are optical minimally invasive approaches based on oxygen-dependent quenching of fluorescence or phosphorescence that can be used to image oxygen tension within retinal vessels and allows for measuring the oxygen concentration (Feenstra, et al., Early Events in Diabetic Retinopathy and Intervention Strategies, 47 (2018); Wanek, et al., Biomedical optics express 2, 2562-2568 (2011)). The disadvantage of this technique is that it cannot provide long-term visualization of hypoxia due to rapid clearance of the injected small molecule dyes from the body. Thus, there is a critical clinical need for an effective, non-destructive method to measure oxygen tension in the tissue microenvironment rather than strictly within retinal blood vessels, and no clinical solution exists to this problem.

### SUMMARY

In embodiments, a room-temperature phosphorescence nanoparticle can include: a solid core including a metal-free organic phosphor embedded in a polymer, wherein the polymer is hydrophobic, glassy, and oxygen-permeable and the metal-free organic phosphor is present in an amount of about 0.1 wt % to about 20 wt %, based on the total weight of the polymer; and a shell surrounding the core, the shell comprising a lipid.

In embodiments, a method of detecting hypoxia in a mammalian subject can include administering to the subject room-temperature phosphorescence nanoparticles in accordance with embodiments disclosed herein, and obtaining phosphorescence images of the mammalian subject.

In embodiments, a method of preparing a room-temperature phosphorescence nanoparticle, can include: adding a first solution including a first solvent, a polymer, and a metal-free organic phosphor to a stirring second solution including a second solvent and a lipid under conditions to form the room-temperature phosphorescence nanoparticle including a solid core including the metal-free organic phosphor embedded in a polymer and a shell surrounding the core including the lipid, wherein the polymer is hydrophobic, glassy, and oxygen-permeable, and the metal-free organic phosphor is present in an amount of about 0.1 wt % to about 20 wt %, based on the total weight of the polymer.

### BRIEF DESCRIPTION OF DRAWINGS

FIG. 1A is a graph of the hydrodynamic size (diameter, nm) distribution of an embodiment of the room-temperature phosphorescence nanoparticles of the disclosure measured by dynamic light scattering (DLS). The inset shows a light-scattering image visualized by Nanoparticle Tracking Analysis, corroborating well-dispersed nanoparticles in aqueous solutions.

FIG. 1B is a graph of the surface zeta potential change of an embodiment of the room-temperature phosphorescence nanoparticles of the disclosure with and without lipid coating.

FIG. 1C is a graph of the steady state photoluminescence excitation, emission, and delayed emission (delayed for 500 μs) spectra of an embodiment of the room-temperature phosphorescence nanoparticles of the disclosure dispersed in Argon (Ar)-purged, anoxic aqueous solution.

FIG. 1D is a graph of the phosphorescence lifetime of an embodiment of the room-temperature phosphorescence nanoparticles of the disclosure in an anoxic aqueous solution monitored at 530 nm (λₑₓ=365 nm).

FIG. 1E is a graph of the temporal stability of an embodiment of the room-temperature phosphorescence nanoparticles of the disclosure stored in Milli-Q water at room temperature. Nanoparticle size and polydispersity were monitored over the course of 13 weeks using DLS.

FIG. 1F is a graph of oxygen sensitivity calibration, wherein the steady state photoluminescence emission of an embodiment of the room-temperature phosphorescence nanoparticles of the disclosure suspended at various O₂ saturation levels (0-21%). λₑₓ=365 nm.

FIG. 1G is a scanning electron microscopy image showing the morphology of an embodiment of the room-temperature phosphorescence nanoparticles of the disclosure. Scale bar: 300 nm.

FIG. 2 are longitudinal phosphorescence photographic images of an embodiment of intravitreal room-temperature phosphorescence nanoparticles of the disclosure in living rabbit retinal hypoxia and control over 7 days (λₑₓ=365 nm, λₑₘ=530 nm).

FIG. 3 is a schematic of an eye with the room-temperature phosphorescence nanoparticles of the disclosure being injected into it and a graph of the average phosphorescence intensity measured from the hypoxic side and the control side. Error bars show the standard deviations of three independent measurements.

FIG. 4 depicts a schematic of an example of a nanoprecipitation method of room-temperature phosphorescence nanoparticles of the disclosure and the illumination of the nanoparticles after the O₂ levels are decreased.

FIG. 5 are longitudinal phosphorescence images of a healthy control rabbit before and up to 17 days after the injection of the room-temperature phosphorescence nanoparticles of the disclosure.

FIG. 6 is a graph of the average phosphorescence intensities from the injection and non-injection side (control) over 17 days. No phosphorescence emission was observed on the phosphorescence images at any time point, since there is no tissue hypoxia.

FIG. 7 are in vivo multimodal fluorescence and phosphorescence images of intravitreal room-temperature phosphorescence nanoparticles of the disclosure and retinal hypoxia in living rabbits.

FIG. 8 is a graph of a dynamic light scattering measurement of an embodiment of the room-temperature phosphorescence nanoparticles of the disclosure fabricated from polystyrene-b-poly(4-vinylpyridine) (“PS4VP”) and DSPE-PEG2000.

FIG. 9 is a graph of the quantification of mean phosphorescence intensities at laser lesions over 7 days comparing an embodiment of the room-temperature phosphorescence nanoparticles of the disclosure and a control.

FIG. 10 are color fundus photography (row c), fluorescence (row d), and phosphorescence (row e) images before and after intravenous injection of 4 mL of an embodiment of the room-temperature phosphorescence nanoparticles of the disclosure at a concentration of 2.5 mg/mL.

FIG. 11 is a graph of body weight increase measured daily for 7 days from three different groups: untreated control and intravenously treated CVO models in White New Zealand and Dutch Belted rabbits, showing no evidence of systematic toxicity through equivalent, appropriate weight gain in both groups: treated with an embodiment of the room-temperature phosphorescence nanoparticles of the disclosure group and untreated control group.

FIG. 12 shows images of H&E staining of tissues obtained 1-month post-intravenous injection of an embodiment of the room-temperature phosphorescence nanoparticles of the disclosure (row b) compared to the untreated control group (row c), demonstrating preserved cellular morphology and nuclei without fragmentation or extracellular debris from dead cells in all of the evaluated organs, including eye, heart, kidney, lung, liver, and spleen. A TUNEL assay analysis is shown (row d) at 1-month post-intravenous injection of an embodiment of the room-temperature phosphorescence nanoparticles of the disclosure. DAPI indicates cell nuclei. Green color stained with FITC evaluates for any apoptotic cells, which are not noted. Scale bar: 75 μm.

FIG. 13 shows in vivo phosphorescence images of the retinal hypoxia acquired at different time points post-injection of an embodiment of the room-temperature phosphorescence nanoparticles of the disclosure in a living rabbit.

FIG. 14 is a graph of hydrodynamic size distribution of an embodiment of the room-temperature phosphorescence nanoparticles of the disclosure (Br6A-IR780-LPS4Br NPs) measured by dynamic light scattering.

FIG. 15 is a collection of images showing CVO model generation and leakage confirmation at day 7 post photocoagulation. Top row images show color fundus photography (left) and fundus autofluorescence (FAF) image (right). Middle and bottom rows exhibit fluorescein angiography (FA) and indocyanine green angiography (ICGA) images, respectively, acquired at different phases: early, middle, and late phase.

FIG. 16 is a collection of images showing in vivo hypoxia visualization of CVO in rabbits. Color fundus photography (row b) of the eye pre- and post-intravenous injection of a nanoparticle including a fluorescent dye, a polymer, and a lipid (IR780-LPS4VP-PEG NPs). White arrows show the local hypoxia induced by laser photocoagulation. Fluorescent images (row c) obtained after injection of 4 mL nanoparticles (2.5 mg/mL). The treated areas were clearly observed from the leakage of nanoparticles at 15 min (arrows). Phosphorescence images (row d) show no signal emission from the NPs.

FIG. 17 is a graph of body weight increase of untreated control and RTP NPs treated RVO model in White New Zealand rabbits via intravitreal injection measured daily for 7 days.

FIG. 18 is a collection of H&E images of tissues after intravitreal injection of an embodiment of the room-temperature phosphorescence nanoparticles of the disclosure (row b) compared to the untreated control group (row c). Scale bar: 100 μm. A TUNEL assay analysis was also done (row d). DAPI (row d) indicates cell nuclei. Green color stained with FITC evaluates for any apoptotic cells, which are not noted. Scale bar: 75 μm.

FIG. 19 is a graph showing steady state excitation (line), emission (line) spectra, and delayed emission spectra (dots) of Se—CO in atactic PMMA (spin-coated, 1 wt % doping concentration) measured at 298K or 78K in anoxic conditions.

FIG. 20 is a graph showing steady state excitation (ssEx) and emission (ssEm) spectra of SeCO—LPS4Br NPs dispensed in water measured at 298K in anoxic condition. Emission curves were recorded with various excitation wavelengths.

FIG. 21 includes images under 400 nm excitation with a Fundus camera of testing of SeCO—IR780-LPS4VP-PEG NPs in live rabbits.

### DETAILED DESCRIPTION

Room-temperature phosphorescence (“RTP”) nanoparticles (“NPs”) in accordance with embodiments of the disclosure can include a solid core having a metal-free organic phosphor embedded in a polymer; and a shell surrounding the core. The shell includes a lipid. In embodiments, the polymer is hydrophobic, glassy, and oxygen-permeable. In embodiments, the metal-free organic phosphor is present in an amount in a range of about 0.1 wt % to about 20 wt %, based on the total weight of the polymer. The room-temperature phosphorescence nanoparticles disclosed herein can optically visualize chorioretinal tissue hypoxia in real-time with high signal-to-noise ratio.

Methods of the disclosure can allow for a one-pot self-assembly protocol that is easily scalable. Methods of the disclosure result in biocompatible nanoparticles that allow the organic phosphors embedded therein to have enhanced biocompatibility and spatiotemporal resolution for in vivo bioimaging. The fabricated room-temperature phosphorescence nanoparticles of the disclosure exhibit long-lived bright RTP with high sensitivity toward oxygen quenching and good long-term stability in vitro, making the room-temperature phosphorescence nanoparticles promising tissue hypoxia imaging agents for pre-clinical studies. The RTP nanoparticles disclosed herein can be advantageously used as a non-destructive method to visualize oxygen tension in chorioretinal tissue rather than in hemoglobin within the retinal vasculature. In embodiments, the RTP nanoparticles can enable quantitative mapping of oxygen gradient and measure the degree of tissue hypoxia with high spatiotemporal resolution.

Conventional fluorescent probes used in the bioimaging space have very short-lived decay time (nanoseconds) and thus are insensitive to oxygen tension change. By contrast, metal-free purely organic phosphors as disclosed herein are an emerging class of room-temperature phosphorescence materials with unique properties, including but not limited to, a large Stokes shift that effectively eliminates the interference of the excitation light source or the background autofluorescence by wavelength-based deconvolution, enabling high signal-to-noise ratio measurements, as well as having a long lifetime (milliseconds to seconds) (Ma, et al., Acc. Chem. Res. 2019, 52 (3), 738-748; Kenry, et al., Nat. Commun. 2019, 10 (1), 2111; Gan, et al., Adv. Funct. Mater. 2018, 28 (51)). Said features endow organic phosphorescence-based sensors numerous advantages over traditional or state of the art optical sensors designed for hypoxia detection and imaging in biological systems. Metal-free purely organic phosphors can advantageously generate a purely organic phosphorescence signal from the long-lived triplet excited state that is highly susceptible to molecular oxygen quenching through triplet energy transfer. (Hirata, et al., Adv. Funct. Mater. 2013, 23 (27), 3386-3397; Vanderkooi, et al., J. Biol. Chem. 1987, 262 (12), 5476-5482; Schulman, et al., J. Phys. Chem. 1977, 81 (20), 1932-1939). Further, unlike conventional inorganic or organometallic-based RTP materials containing precious rare-earth and transition metals, and exhibiting toxicity or stability issues in bio-applications (Rai et al., Biomedical Applications of Metals. Springer: Cham, 2018; p xviii, 325 pages), purely organic phosphors are more cost-effective, robust, and biocompatible.

Advantages of the room-temperature phosphorescence nanoparticles of the present disclosure can include color and oxygen sensitivity that can be readily customized by using different organic phosphors and polymer matrices, depending on the intended use. Further advantages can include a phosphorescent signal with long lifetime and high quantum efficiency (bright green for Br6A) that can afford high signal-to-noise ratio for bioimaging, eliminating the interference of background autofluorescence typically generated by tissue; as well as, the size of NPs can be tuned, leading to high-resolution, minimally invasive oxygen mapping in vivo. Advantages of the method of preparing the room-temperature phosphorescence nanoparticles of the present disclosure include facile synthetic method and engineering approach reduces the burden of functionalization through lengthy chemical synthesis.

In conventional systems, bright organic RTP is generally achieved through doping in rigid hosts such as solid-state crystalline structures or rigid polymer films to suppress molecular motion. Kwon, et al., Nat. Commun. 2015, 6, 8947). However, using said rigid hosts limit the practical applicability of organic phosphors toward in vivo hypoxia detection because good oxygen diffusivity, excellent aqueous dispersibility, and biocompatibility also need to be integrated into the material design and processing. Consequently, despite the great potential, there are very few organic RTP material systems exploited as in vivo oxygen sensors (DeRosa, et al., ACS Sens. 2016, 1 (11), 1366-1373; Samonina-Kosicka, et al., Macromol. Rapid Commun. 2015, 36 (7), 694-699; Kersey, et al., ACS Nano 2010, 4 (9), 4989-4996; Zhang, et al., Nat. Mater. 2009, 8 (9), 747-751).

### Room-Temperature Phosphorescence Nanoparticles

Disclosed herein are room-temperature phosphorescence nanoparticles. The room-temperature phosphorescence nanoparticles include a solid core having a metal-free organic phosphor embedded in a polymer and a shell surrounding the core. The shell includes a lipid. As used herein, the phrase “embedded in a polymer” refers to homogenous or substantially homogenous distribution of a substance throughout the polymer matrix. In embodiments, the polymer is hydrophobic, glassy, and oxygen-permeable. In embodiments, the metal-free organic phosphor is present in an amount of about 0.1 wt % to about 20 wt %, based on the total weight of the polymer.

Metal-free organic phosphors are organic compounds including a first portion comprising a carbonyl group bonded to an aromatic group or an aromatic group having extended π-conjugation; a second portion comprising a halogen atom bonded to an aromatic group or an aromatic group having extended π-conjugation; and an intermolecular non-covalent interaction between the oxygen of the carbonyl group and the halogen atom, wherein the distance between the oxygen of the carbonyl group and the halogen atom is less than the van der Waals radii of the oxygen of the carbonyl group and the halogen atom. Various metal-free organic phosphors can be used in the nanoparticles of the disclosure, such as those disclosed in U.S. Pat. No. 8,525,131, the disclosure of which is incorporated by reference in its entirety.

The metal-free organic phosphor as used in embodiments of the disclosure can be selected to have a long emission lifetime, such as having an emission lifetime of about 1 microseconds to about 100 seconds or more. In embodiments, the metal-free organic phosphor can be one or more of

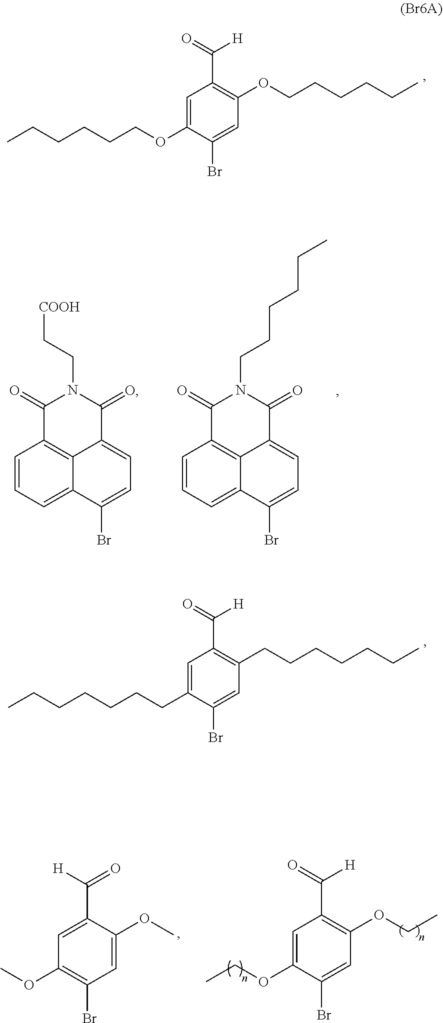

wherein n can be 4, 6, or 7,

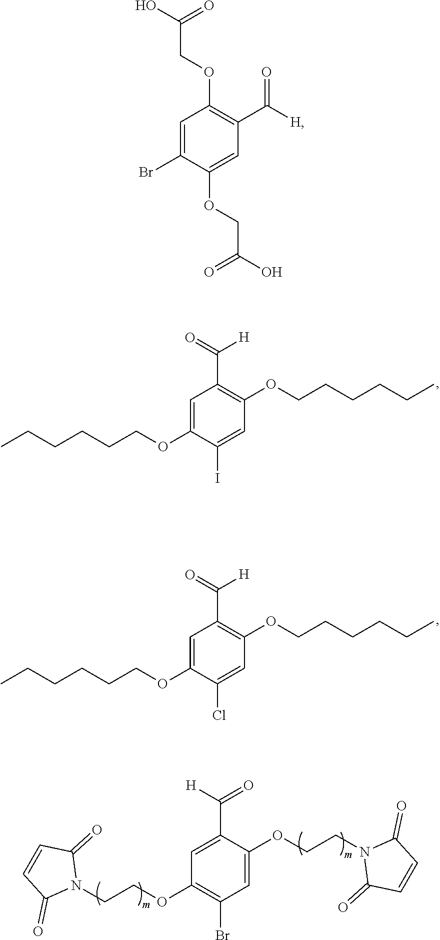

wherein m can be 1, 2, or 3,

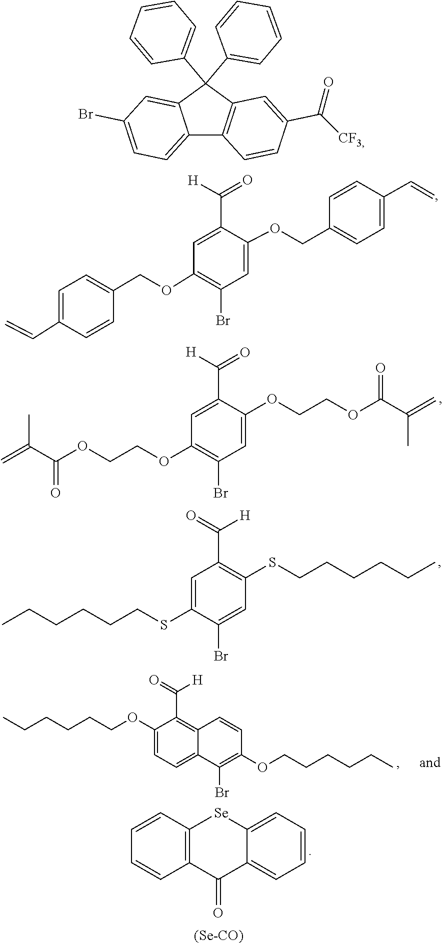

In embodiments, the metal-free organic phosphor is Br6A. In embodiments, the metal-free organic phosphor can include a metal-free organic phosphor that has an emission lifetime of at least 1 microsecond. In embodiments, the metal-free organic phosphor can include a metal-free organic phosphor that has an emission lifetime in a range of about 1 microsecond to about 100 seconds, about 1 millisecond to about 10 seconds, about 1 millisecond to about 1 second, or about 10 milliseconds to about 1 second.

The polymer can be any polymer that is one or more of hydrophobic, glassy, and oxygen-permeable. As used herein, the term “glassy” refers to a polymer that is amorphous with a glass transition temperature above room temperature. As used herein, the term “oxygen permeable” refers to a polymer through which oxygen can diffuse.

In embodiments, the polymer can include a polystyrene. In embodiments, the polymer is a polystyrene. In embodiments, the polystyrene can include a polystyrene homopolymer, a halogenated polystyrene, a polystyrene copolymer, or a combination thereof. In embodiments, the polymer is a polystyrene copolymer. In embodiments, the polystyrene copolymer can include a vinyl pyridine modification. In embodiments, the polystyrene copolymer is polystyrene-b-poly(4-vinylpyridine) which has a structure of:

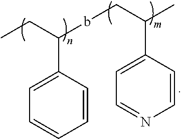

In embodiments, the polymer can include a halogenated polystyrene. In embodiments, the polymer is a halogenated polystyrene. In embodiments, the halogenated polystyrene comprises one or more of fluorine, bromine, chlorine, and iodine. In embodiments, the halogenated polystyrene comprises bromine. In embodiments, the halogenated polystyrene is poly (2-bromostyrene), poly (3-bromostyrene), or poly (4-bromostyrene). In embodiments, the halogenated polystyrene is poly (4-bromostyrene).

The polymer can be selected herein to have optical inertness, rigidity and oxygen permeability. Selection and use of such a polymer in the nanoparticles herein can aid in achieving bright and sensitive RTP for chorioretinal tissue oxygen detection.

The polymers can also be selected herein to have sufficient rigidity to effectively suppress the vibrational energy dissipation of the metal-free phosphor for bright RTP emission.

Further, the polymer can be selected to have a desired oxygen permeability to allow oxygen to permeate to the embedded organic phosphor, which can then respond to oxygen tension change in the surrounding environment in real-time. It has been advantageously found that styrene-based polymers have desirable rigidity and proper oxygen permeability for use in the present nanoparticles. Styrene-based polymers have been exploited in sensors for dissolved oxygen detection Yu, et al., Angew. Chem. Int. Ed. 2017, 56 (51), 16207-16211; Siracusa; Int. J. Polym. Sci. 2012; Miller et al., Trends Food Sci. Technol. 1997, 8 (7), 228-237). Additionally, it has been found that the brominated polystyrene matrix (e.g., poly (4-bromostyrene)) can enhance the RTP emission of Br6A through external halogen bonding. The halogen bonding between the oxygen atom of the carbonyl group of Br6A and the neighboring bromine atom of PS4Br can further suppress the vibrational energy dissipation meanwhile enhancing spin-orbit coupling and intersystem crossing of Br6A (Bolton et al., Nat. Chem. 2011, 3 (3), 205-210).

The polymer can have any suitable molecular weight (Mw). In embodiments, the polymer's molecular weight (Mw) is in a range of about 1000 to about 300,000. The polymer molecular weights provided herein are all in units of Dalton. For example, in embodiments, the polymer can be a polystyrene with a molecular weight (Mw) in a range of about 10,000 to about 300,000, or about 10,000 to about 200,000, or about 5000 to about 150,000, or about 10,000 to about 125,000, or about 10,000 to about 100,000, or about 20,000 to about 60,000, or about 30,000 to about 100,000, or about 30,000 to about 50,000, or about 35,000 to about 45,000.

Any suitable lipid or lipid combination can be used for the shell. The lipid-based shell enables the nanoparticle to be dispersible in an aqueous phase as well as enhancing the biocompatibility of the nanoparticles for in vivo applications. The one or more lipids can have a net positive charge (i.e., a cationic lipid), a net negative charge (i.e., an anionic lipid), no charges (i.e, a nonionic lipid), or an equal number of positive and negative charges (i.e., a zwitterionic lipid). In embodiments, the lipid is amphiphilic. In embodiments, the lipid is anioinic. In embodiments, the lipid is both amphiphilic and anionic advantageously increasing the stability of the RTP nanoparticles in water by preventing aggregation through electrostatic repulsion or hydration, particularly at concentrated states and for long-term storage.

In embodiments, the lipid is a phospholipid. The structure of a phospholipid generally comprises a hydrophobic tail and a hydrophilic head group and is amphiphilic in nature. Most phospholipids contain a diglyceride, a phosphate group, and a simple organic molecule such as choline; one exception to this rule is sphingomyelin, which is derived from sphingosine instead of glycerol. Phospholipids include, without limitation, diacylglycerides and phosphosphingolipids. Non-limiting examples of diacylglycerides include a phosphatidic acid (phosphatidate) (PA), a phosphatidylethanolamine (cephalin) (PE), a phosphatidylcholine (lecithin) (PC), a phosphatidylserine (PS), and a phosphoinositide including phosphatidylinositol (PI), phosphatidylinositol phosphate (PIP), phosphatidylinositol bisphosphate (PIP2), and phosphatidylinositol triphosphate (PIP3). Non-limiting examples of phosphosphingolipids include a ceramide phosphorylcholine (sphingomyelin) (SPH), ceramide phosphorylethanolamine (sphingomyelin) (Cer-PE), and ceramide phosphorylglycerol. The purpose of phospholipid coating on the NP surface was manifold. As the main components of the cellular membrane, phospholipids have excellent biocompatibility (Li, J. et al. Asian J. Pharm. Sci. 10, 81-98 (2015)). Its amphiphilic and anionic structure can help increase the NPs' stability in water by preventing their aggregation through electrostatic repulsion or hydration (Shah et al., I. in Lipid nanoparticles: Production, characterization and stability Springer briefs in pharmaceutical science & drug development 75-97 (Springer, Cham, 2015)), particularly at concentrated states and for long-term storage.

In embodiments, the lipid can include one or more of a phospholipid as identified above, 1,2-dimyristoyl-sn-glycero-3-phosphate (DMPA), 1,2-distearoyl-sn-glycero-3-phosphoethanolamine-N-[carboxy(polyethylene glycol)-2000] (DSPE-PEG2000) or a salt thereof, 1,2-distearoyl-sn-glycero-3-phosphoethanolamine-N-[methoxy(polyethylene glycol)-2000] or a salt thereof, the ammonium salt of 1,2-distearoyl-sn-glycero-3-phosphoethanolamine-N-[methoxy(polyethylene glycol)-2000], and polyethylene glycol. In embodiments, the lipid can include 1,2-distearoyl-sn-glycero-3-phosphoethanolamine (DSPE) bonded to polyethylene glycol-2000, and optionally the PEG-2000 is bonded to an end group. In general, the end group can be any suitable end group to one of ordinary skill in the art. In embodiments, the end group can include maleimide, nitrobenzofurazan, biotin, carboxylic acid (—COOH), thiol (—SH), fluorescein, succinimidyl ester (NHS), pyridyldithiol propionate (PDP), primary amine (—NH₂), hydroxyl (—OH), azide, folate, halide, or the like.

Optionally, the room-temperature phosphorescence nanoparticles can further include a fluorescent dye. Advantageously, the room-temperature phosphorescence nanoparticles disclosed herein can include a fluorescent dye in the solid core, which can enable the nanoparticles herein to achieve dual phosphorescence and fluorescence imaging. Since fluorescent signals are not affected by oxygen tension change, co-localizing the fluorescence and phosphorescence signals using different wavelength channels enables the tracking of the post-injection distribution of RTP nanoparticles.

The fluorescent dye can be any suitable fluorescent dye. In embodiments, the fluorescent dye can be any suitable fluorescent dye having similar solubility/hydrophobicity as the metal-free organic phosphor and the polymer disclosed herein. In embodiments, the fluorescent dye can include one or more of IR-780 and nile red. IR-780 has a structure of

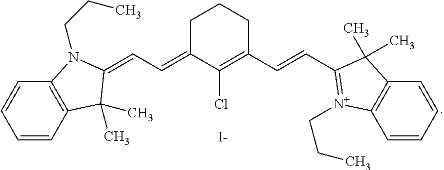 [SMILES: CCCN1/C(=C/C=C2\CCCC(/C=C/C3=[N+](CCC)c4ccccc4C3(C)C)=C2Cl)C(C)(C)c2ccccc21 ; [I-]]

Nile red has a structure of

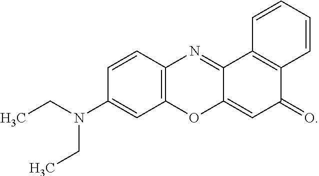 [SMILES: CCN(CC)c1ccc2nc3c4ccccc4c(=O)cc-3oc2c1]

In embodiments, the fluorescent dye can be embedded in the polymer.

In embodiments, the room-temperature phosphorescence nanoparticle includes a solid core including Br6A embedded in 4-bromopolystyrene, and a shell including DMPA. In embodiments, the room-temperature phosphorescence nanoparticle includes a solid core including Br6A and IR-780 embedded in 4-bromopolystyrene, and a shell including DMPA. In embodiments, the room-temperature phosphorescence nanoparticle includes a solid core including Br6A embedded in polystyrene-b-poly(4-vinylpyridine), and a shell including DSPE-PEG2000. In embodiments, the room-temperature phosphorescence nanoparticle includes a solid core Br6A and IR-780 embedded in polystyrene-b-poly(4-vinyl pyridine), and a shell including DSPE-PEG2000.

The room-temperature phosphorescence nanoparticles disclosed herein have a diameter in a range of about 1 nm to about 1000 nm. In embodiments, the room-temperature phosphorescence nanoparticles disclosed herein have a diameter in a range of about 10 nm to about 500 nm, or about 10 nm to about 300 nm, about 1 nm to about 200 nm, about 10 nm to about 200 nm, about 10 nm to about 150 nm, about 1 nm to about 100 nm, about 50 nm to about 200 nm, or about 100 nm to about 250 nm, or about 120 nm to about 200 nm, or about 130 nm to about 180 nm, or about 150 nm to about 175 nm, or less than about 100 nm. For example, the room-temperature phosphorescence nanoparticles disclosed herein have a diameter of about 10 nm, about 20 nm, about 30 nm, about 40 nm, about 50 nm, about 75 nm, about 100 nm, about 125 nm, about 150 nm, about 160 nm, about 165 nm, about 170 nm, about 175 nm, about 200 nm, about 250 nm, about 300 nm, about 400 nm, or about 500 nm.

The RTP nanoparticles disclosed herein can be included in a formulation for administration through intravenous injection, topical administration, and local injection. Topical administration can include formulations such as ophthalmic drops. Local injection can be subconjunctival, intraocular, or intravitreal. In embodiments, the formulation for intravenous injection is formulated such that the RTP NPs can be administered in a minimally invasive way to tissue for in vivo hypoxia imaging. In embodiments, the formulation for intravenous injection is formulated such that the RTP NPs can be administered in a minimally invasive way to chorioretinal tissue for in vivo hypoxia imaging. In embodiments, the RTP nanoparticles are formulated to have an average diameter size of 10-150 nm to reduce reticuloendothelial system (RES) clearance and evade the 5 nm renal filtration cut off. In embodiments, the RTP nanoparticles are formulated to have an average diameter size of less than about 100 nm. In embodiments, the formulation is designed with RTP nanoparticles having a diameter size of less than about 100 nm so that the nanoparticles can more effectively extravasate and accumulate at occluded sites, leveraging the enhanced permeability and retention (EPR) effect, as well as having higher diffusivity, allowing for more efficient penetration into the hypoxic chorioretinal tissue. In embodiments, the RTP nanoparticles of the formulation have an average diameter size of about 40 nm to about 50 nm. In embodiments, the formulation for intravenous injection or local injection can include a solvent that the RTP nanoparticles are suspended in. In embodiments, the solvent comprises water. Use of other pharmaceutically acceptable solvents are also contemplated herein.

### Methods of Preparing Room-Temperature Phosphorescence Nanoparticles

In accordance with embodiments, a method of preparing the room-temperature phosphorescence nanoparticles can include includes adding a first solution including a first solvent, the polymer, and the metal-free organic phosphor to a stirring second solution including a second solvent and the lipid. Nanoprecipitation of the nanoparticles of the disclosure results from the admixing of the first and second solutions. Nanoprecipitation is a simple method which can produce homogenous nanoparticles rapidly on a large scale by utilizing a sharp solubility change of host materials in miscible dissimilar solvents (Schubert et al., Soft Matter, 2011, 7 (5), 1581-1588).

The first solvent is intended to dissolve the polymer and optionally the metal-free organic phosphor. In embodiments, the first solvent can include an aprotic organic solvent or combination of aprotic organic solvents. Aprotic solvents can include, but are not limited to, tetrahydrofuran (THF), acetonitrile, ethyl acetate, dimethylformamide (DMF), acetone, 1,4-dioxane, chlorobenzene, nitromethane, diethyl ether, triethyl amine, and dimethyl sulfoxide (DMSO). In embodiments, the aprotic organic solvent includes one or more of tetrahydrofuran (THF), acetonitrile, ethyl acetate, dimethylformamide (DMF), acetone, and dimethyl sulfoxide (DMSO). In embodiments, the first solvent includes THF. In embodiments, the first solvent is THF.

The first solution can include the polymer in an amount of about 0.1 mg/mL to about 100 mg/mL. For example, the polymer can be included in the first solution in an amount of about 0.1 mg/mL to about 75 mg/mL, or about 0.1 mg/mL to about 50 mg/mL, or about 1 mg/mL to about 25 mg/mL, or about 1 mg/mL to about 20 mg/mL, or about 1 mg/mL to about 15 mg/mL, or about 8 mg/mL to about 12 mg/mL. In embodiments, the first solution can include a polymer in an amount of about 1 mg/mL to about 15 mg/mL, such as 10 mg/mL.

The first solution can include the metal-free organic phosphor in an amount of about 0.1 wt % to about 50 wt % based on the total amount of polymer in the first solution. For example, the metal-free organic phosphor can be included in the first solution in an amount of about 0.1 wt % to about 45 wt %, or about 1 wt % to about 35 wt %, or about 1 wt % to about 25 wt %, or about 1 wt % to about 15 wt %, or about 2 wt % to about 10 wt %, based on the total amount of polymer. In embodiments, the first solution can include the metal-free organic phosphor in an amount of about 2 wt % to about 10 wt %, such as about 5 wt % based on the total amount of polymer.

The second solvent is intended to dissolve the lipid as well as to precipitate the polymer when mixed with the first solvent. In embodiments, the second solvent can include a protic organic solvent, water, or both. Protic organic solvents can include, but are not limited to, methanol, ethanol, isopropyl alcohol, 1-butanol, 2-butanol, t-butyl alcohol, and acetic acid. In embodiments, the second solvent is a protic organic solvent. In embodiments, the second solvent is water. In embodiments, the second solvent includes both a protic organic solvent and water. In embodiments, the protic organic solvent can be included in the second solvent in an amount of about 1 wt % to about 50 wt %, based on the total amount of water. For example, the protic organic solvent can be in an amount of about 1 wt % to about 25 wt %, or about 1 wt % to about 15 wt %, or about 1 wt % to about 10 wt %, or about 1 wt % to about 5 wt %, based on the total amount of water. In embodiments, the second solvent includes water and ethanol.

The lipid can be included in the second solution in an amount of about 0.001 mg/mL to about 3 mg/mL. For example, the lipid can be in the second solution in an amount of about 0.01 mg/mL to about 1.5 mg/mL, or about 0.01 mg/mL to about 1 mg/mL, or about 0.05 mg/mL to about 0.5 mg/mL, or about 0.01 mg/mL to about 0.6 mg/mL or about 0.1 mg/mL to about 0.2 mg/mL.

In embodiments, after the first and second solutions are stirred together, the first and second solutions can be sonicated for about 1 minute to about 2 hours. For example, the first and second solutions can be sonicated for about 1 minute to about 1 hour, or about 1 minute to about 45 minutes, or about 1 minutes to about 30 minutes, or about 1 minute to about 15 minutes, or about 5 minutes to about 15 minutes.

It has been found that the inclusion of the lipid allows for more homogenous dispersion of the polymer in the aqueous phase for formation of discrete particles through the nanoprecipitation process. The direct injection of the first solution having polymer at high concentrations (e.g., 10 mg/mL PS4Br) to the aqueous outer phase without DMPA lipid resulted in massive aggregation instead of discrete NPs. Without the presence of a lipid, stable Br6A-PS4Br NPs could only be produced only at a low polymer concentration (1 mg/mL), which lead to very dilute NP samples. Without intending to be bound by theory, it is believed that the addition of lipids allowed for homogeneous dispersion of the polymer in the aqueous phase by serving as a surfactant, which reduces the surface and interfacial tension and stabilizes the polymer-water interface during the nanoprecipitation process. (Yamada, H. et al., ACS App. Mater. Interfaces 6, 3491-3500 (2014); Morsy, S. M. I., Int. J. Curr. Microbiol. App. Sci. 3, 237-260 (2014)).

Further provided herein are methods of detecting hypoxia and/or determining oxygen tension in a mammalian subject administering to the subject the RTP nanoparticles disclosed herein, and obtaining phosphorescence images of the mammalian subject. In embodiments, methods of detecting hypoxia and/or determining oxygen tension in a mammalian subject can further comprise obtaining color fundus photography, phosphorescence and fluorescence images of the mammalian subject. In embodiments, obtaining the phosphorescence images can occur in a range of about 1 minute to about 7 days after injection. In embodiments, obtaining the phosphorescence images can occur at about 1 minute, about 5 minutes, about 10 minutes, about 15 minutes, about 30 minutes, about 45 minutes, about 1 hour, about 2 hours, about 3 hours, about 4 hours, about 5 hours, about 6 hours, about 12 hours, about 24 hours or more after injection.

In embodiments, the RTP nanoparticles are administered via intravenous injection, topical administration (e.g., ophthalmic drops), and local injection (e.g., subconjunctival, intraocular, or intravitreal). In embodiments, the RTP nanoparticles are injected into the mammalian subject in a suspension. In embodiments, the suspension comprises water and the RTP nanoparticles. In embodiments, the suspension can include an excipient. In embodiments, the excipient includes glucose. In embodiments, the suspension comprises the RTP nanoparticles in a concentration in a range of about 0.01 mg/mL to about 25 mg/mL. For example, the suspension comprises the RTP nanoparticles in a concentration in a range of about 0.1 mg/mL to about 20 mg/mL, or about 0.5 mg/mL to about 10 mg/mL, or about 1 mg/mL to about 5 mg/mL, or about 1.5 mg/mL to about 3 mg/mL (e.g., 2.5 mg/mL). In embodiments wherein the RTP nanoparticles are administered intraocularly, the suspension can be administered in a volume of about 0.2 mL or less. In embodiments, the suspension can be administered in a volume in a range of about 0.001 mL to about 0.2 mL, or about 0.01 mL to about 0.1 mL.

In embodiments, the mammalian subject is a human. In embodiments, the mammalian subject has tissue hypoxia. In embodiments, the mammalian subject has chorioretinal hypoxia.

Further provided herein are uses of the RTP nanoparticles disclosed herein for detecting hypoxia in a mammalian subject. The hypoxia can be detected by administering to the subject the RTP nanoparticles disclosed herein, and obtaining phosphorescence images of the mammalian subject. In embodiments, the mammalian subject is a human. In embodiments, the mammalian subject has tissue hypoxia. In embodiments, the mammalian subject has chorioretinal hypoxia.

### EXAMPLES

Materials and Methods: Reagents used to synthesize the organic phosphor Br6A were purchased from Millipore Sigma. Phospholipids 1,2-dimyristoyl-sn-glycero-3-phosphate (DMPA) and 1,2-distearoyl-sn-glycero-3-phosphoethanolamine-N-[carboxy(polyethylene glycol)-2000] (DSPE-PEG2000) were purchased from Avanti Polar Lipids. Polystyrene-b-poly(4-vinylpyrindine) (PS4VP) was purchased from Polymer Source. Other chemicals and materials such as IR-780 iodide, poly(4-bromostyrene) (PS4Br, M, ˜65,000), solvents, and Amicon@ Ultra-4 centrifugal filters (MWCO 30 kD) were also purchased from Millipore Sigma. Malvern Panalytical folded capillary zeta cells were purchased from Fisher Scientific.

### Example 1: Preparation of Lipid-Polymer Hybrid Nanoparticles

The metal-free organic phosphor, Br6A was synthesized according to a published procedure from Bolton et al., Nat. Chem. 2011, 3 (3), 205-210, and its purity was confirmed by ¹H NMR. The lipid-polymer hybrid NPs containing the organic phosphor were fabricated by a single-step nanoprecipitation method. To prepare Br6A-LPS4Br NPs, phospholipid DMPA was dispersed into 4 wt % ethanol-water solution at a concentration of 0.15 mg/mL (15 wt % to PS4Br) as the aqueous outer phase. The dispersion was then heated up to 65° C. until the solution became completely transparent to ensure that all the lipids were in the liquid phase. A stock solution of 10 mg/mL PS4Br mixed with Br6A (5 wt % to PS4Br) was prepared in tetrahydrofuran (THF) as the organic internal phase. Then 1 mL of this THF mixture solution was rapidly injected into the aqueous outer phase with vigorous stirring. The resulting suspension was subsequently sonicated for 10 min using a bath sonicator (Bransonic 2510-DTH) at a frequency of 42 kHz and power of 100 W. It was then further stirred at room temperature for 30 h to allow for the complete evaporation of organic solvents. Purification was done by washing the NPs in Milli-Q water 3 times using Amicon® Ultra-4 centrifugal filters with a molecular weight cutoff of 30 kD. The purified Br6A-LPS4Br NPs were re-suspended in Milli-Q water to make 2.5 mg/mL stock concentration. The fluorescent dye co-encapsulated Br6A-IR780-LPS4Br NPs were synthesized using a similar procedure except when mixing PS4Br with organic phosphor Br6A, IR-780 iodide (stock solution prepared in acetone at 1 mg/mL) was also added at 2 wt % to PS4Br.

Preparation of lipid-polymer hybrid organic RTP NPs for intravenous injection: To prepare Br6A-IR780-LPS4VP-(DSPE-PEG2000) NPs for intravenous administration, PS4VP was used as the polymer matrix instead of PS4Br, and DSPE-PEG2000 (30 wt % to PS4VP) was used as the phospholipid instead of DMPA. To make 1 mL of the organic internal phase containing polymer, organic phosphor (5 wt % to polymer), and fluorescent dye (2 wt % to polymer), 400 μL THF was used to dissolve 10 mg of PS4VP, followed by adding 100 μL of Br6A stock in THF at 5 mg/mL and 200 μL of IR-780 iodide stock in acetone at 1 mg/mL. Then 300 μL of acetone was added to the mixture to adjust the volume ratio of THF and acetone (1:1). The rest of the steps were followed according to the nanoprecipitation procedure described above.

Characterization of lipid-polymer hybrid phosphorescent nanoparticles: Hydrodynamic size (diameter, nm), PDI, and surface charge ((potential, mV) of the lipid-polymer hybrid NPs were measured in Milli-Q water on Malvern Zetasizer Nano ZSP (Model number: ZEN5600) using disposable cuvettes and folded capillary zeta cells, respectively. All measurements were conducted at a backscattering angle of 173⁰ (NIBS default) at 25° C. The average hydrodynamic diameters of NPs are reported using the peak means of size distribution plots by intensity. Particle size and morphology were further examined by SEM (Thermo Fisher Nova 200 Nanolab) after carbon coating. Photophysical properties including steady state emission and excitation spectra, delayed emission, lifetime, and absolute quantum yield were measured using a Photo Technologies International (PTI) QuantaMaster spectrofluorometer equipped with an integrating sphere. Anoxic aqueous suspensions of RTP NPs were prepared by bubbling argon gas through sample solutions placed in rubber septum-capped quartz cuvettes for 30 min. Then to achieve various pO₂ (0-21%) in the aqueous suspension, different amounts of air were refilled back into the anoxic sample. All experiments were performed in triplicate from freshly prepared samples (n=3). Data are expressed as the mean±standard deviation.

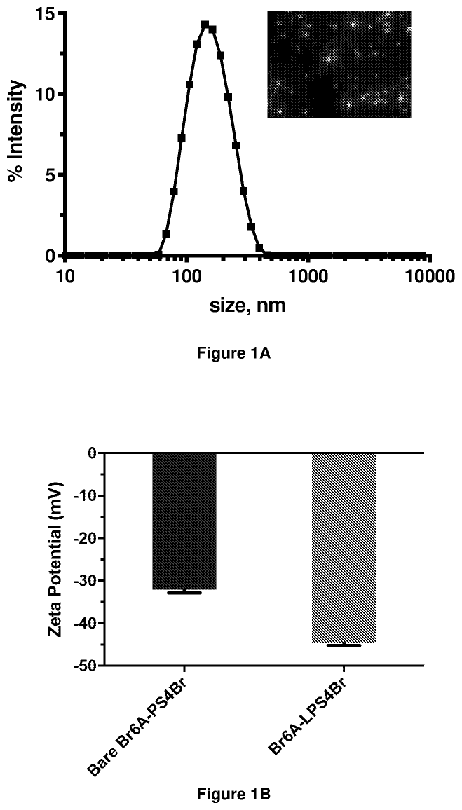

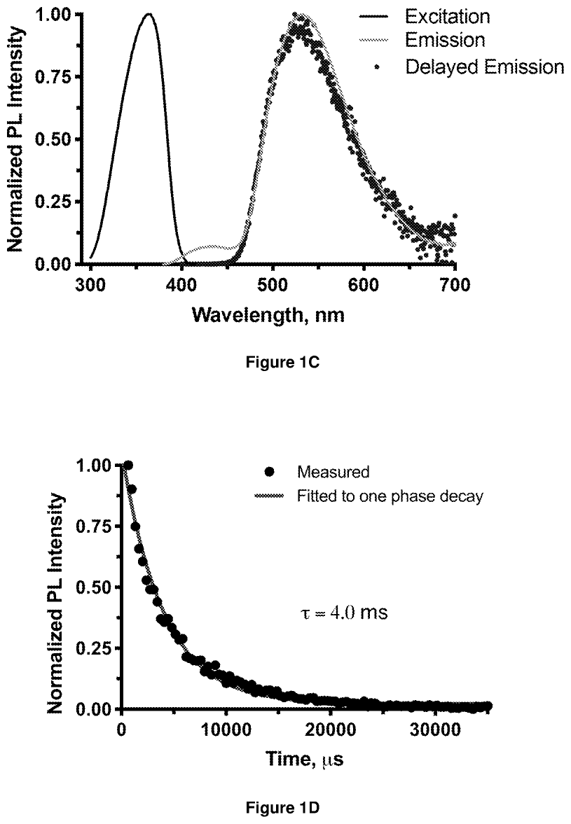

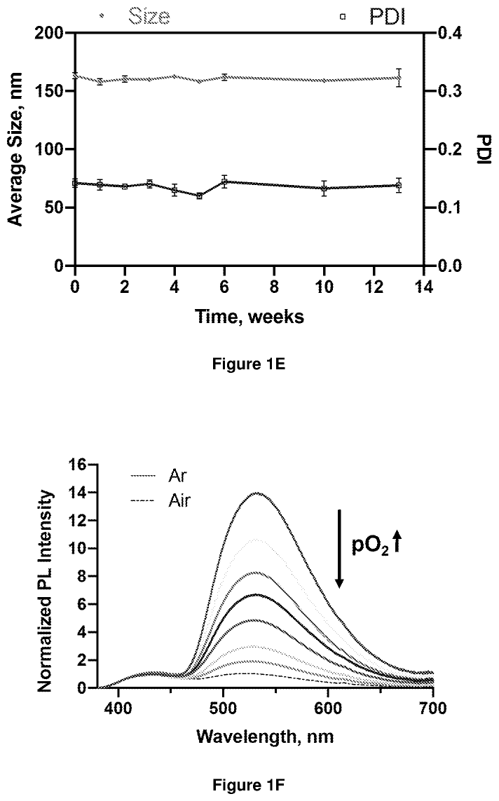

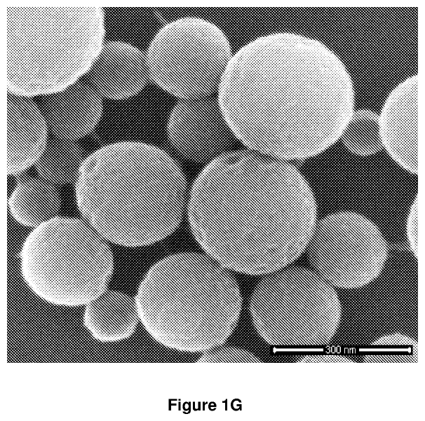

Dynamic light scattering (DLS) data indicated fairly narrow-dispersed Br6A-LPS4Br NPs with an average hydrodynamic diameter of 163.9±2.7 nm (mean±S.D., n=3) and polydispersity index (PDI) of 0.134±0.008 (mean±S.D., n=3) in Milli-Q water (FIG. 1A). The inset in FIG. 1A shows a light-scattering image visualized by Nanoparticle Tracking Analysis, corroborating well-dispersed nanoparticles in aqueous solutions. The more negative zeta potential (ξ) of the resulting Br6A-LPS4Br (−44.7±0.5 mV) in comparison to Br6A-PS4Br (−32.1±0.8 mV) suggested the successful coating of the negatively charged DMPA lipid on the NP surface (FIG. 1B). A comparison of the surface zeta potential change to bare Br6A-PS4Br nanoparticles and Br6A-LPS4Br exhibited a relatively more negative value, indicating its surface was successfully coated with anionic DMPA (FIG. 1B). Particle size and Br6A-LPS4Br nanoparticles spherical shape were further confirmed by scanning electron microscope, scale bar: 300 nm (SEM, FIG. 1G). Temporal stability of Br6A-LPS4Br nanoparticles stored in Milli-Q water at room temperature was tested. Nanoparticle size and polydispersity were monitored over the course of 13 weeks using DLS. No obvious aggregation was observed, suggesting long-term stability. All error bars indicate S.D. (n=3) (FIG. 1E). Steady state photoluminescence excitation, emission, and delayed emission (delayed for 500 μs) spectra of Br6A-LPS4Br nanoparticles dispersed in Argon (Ar)-purged, anoxic aqueous solution is shown in FIG. 1C. Phosphorescence lifetime of the nanoparticles in an anoxic aqueous solution monitored at 530 nm (λₑₓ=365 nm) is shown in FIG. 1D. Photophysical properties of Br6A-LPS4Br nanoparticles confirmed that the green, 530 nm emission is indeed of phosphorescent nature. FIG. 1F shows the oxygen sensitivity calibration of the Br6A-LPS4Br nanoparticles. Steady state photoluminescence emission of Br6A-LPS4Br nanoparticles suspension was taken at various O₂ saturation levels (0-21%), λₑₓ=365 nm.

The colloidal stability of the Br6A-LPS4Br nanoparticles is an important criterion for their long-term in vivo oxygen-sensing applications. The temporal storage stability of Br6A-LPS4Br NPs was studies by monitoring the change in their size and polydispersity over 13 weeks. The stock solution (2.5 mg/mL) was stored in Milli-Q water under ambient conditions. There was no sign of aggregation of the RTP NPs suspension over 13 weeks, as shown in FIG. 1D. Therefore, this simple one-step nanoprecipitation method is easy to scale up for robust production of lipid-stabilized, polymer-supported organic RTP NPs.

The RTP nature of the emission from the fabricated Br6A-LPS4Br NPs was then confirmed. As expected, the NPs aqueous suspension exhibited bright green emission under 365 nm excitation after the removal of dissolved oxygen by argon purging. Steady state photoluminescence spectroscopy data confirmed the excitation and emission spectra maxima at 365 nm and 530 nm, respectively. The small shoulder peak around 425 nm corresponds to the fluorescence emission of Br6A. The gated emission spectrum acquired after a 500 μs delay well-overlapped with the steady state emission maxima at 530 nm, which indicated that the green emission is indeed of phosphorescent nature rather than fluorescent (FIG. 1C). Lifetime (T) measurement of the green emission monitored at 530 nm resulted in 4.0 milliseconds (ms) (FIG. 1D), further corroborating the phosphorescent emission. Quantum efficiency of Br6A-LPS4Br NPs in anoxic aqueous suspension Φₚ (455-675 nm) was measured to be 16.9±3.0%. Therefore, these photophysical characteristics of Br6A-LPS4Br NPs are in good agreement with those of Br6A in crystalline state (Bolton et al., Nat. Chem. 3, 205-210 (2011)) or embedded in isotactic poly(methyl methacrylate) film (Lee, D. et al., J. Am. Chem. Soc. 135, 6325-6329 (2013)) reported previously, indicating the successful inclusion of Br6A into the NP matrix by nanoprecipitation method. The total emission intensity of Br6A-LPS4Br NPs aqueous suspension was tested with various partial pressures of oxygen (pO₂ from 0-21%). The green phosphorescence emission was highly responsive to small changes in pO₂, with gradually quenched phosphorescence signal as the pO₂ increased, whereas the fluorescence emission at 420 nm remained the same (FIG. 1F). This confirmed the hypothesis that the phosphorescence emission of Br6A-based RTP NPs can be applied for sensitive hypoxia detection. Though measurements with more precise control over pO₂ would be needed to calculate the hypoxia detection range, it is reasonable to estimate that these RTP NPs will be able to distinguish differences of approximately 3% in pO₂, and to determine different degrees of tissue ischemia. In addition, the optical stability of Br6A-LPS4Br NPs was studied and it was found that the total emission intensity of the NP suspension upon Argon purging remained the same after 13 weeks.

### Example 2—Se—Co Phosphor Containing Lipid-Polymer Hybrid Nanoparticles

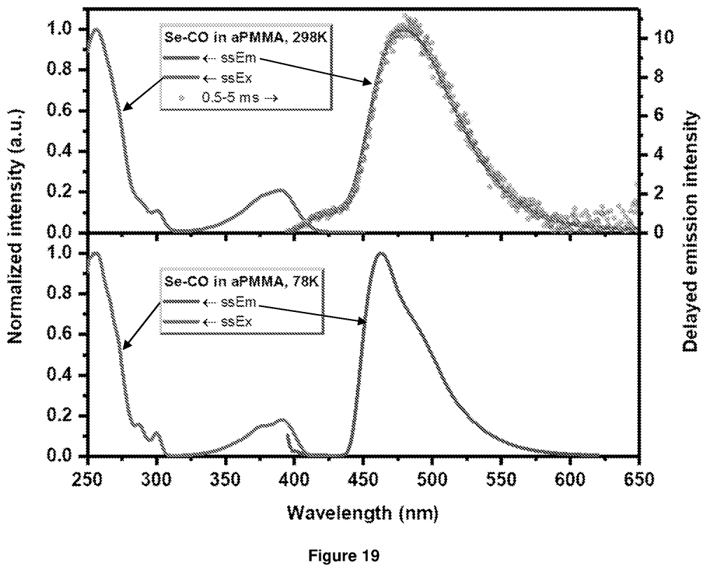

The Se—CO phosphor has been demonstrated to exhibit bright blueish-green room temperature phosphorescence emissions when doped in PMMA (spin-cased, 1 wt % doping concentration). Shao, W. et al., Heavy Atom Oriented Orbital Angular Momentum Manipulation in Metal-Free Organic Phosphors. Preprint available at https://www.researchsquare.com/articles/rs-435553/v1. FIG. 19 is a graph, which shows steady state excitation (line), emission (line) spectra, and delayed emission spectra (dots) of the Se—CO in atactic PMMA measured at 298K or 78K in anoxic conditions. The Se—CO doped in PMMA was demonstrated to have a phosphorescence emission with 792% quantum yield and lifetime of 1.82 ms, measured in anoxic conditions. Based on the excitation spectra of Se—CO, it is capable of being excited with >400 nm light.

Lipid-polymer hybrid NPs containing the Se—CO as the organic phosphor in accordance with the disclosure were fabricated by the single-step nanoprecipitation method of Example 1. To prepare SeCO—LPS4Br NPs, phospholipid DMPA was dispersed into 4 wt % ethanol-water solution at a concentration of 0.15 mg/mL (15 wt % to PS4Br) as the aqueous outer phase. The dispersion was then heated up to 65° C. until the solution became completely transparent to ensure that all the lipids were in the liquid phase. A stock solution of 10 mg/mL PS4Br mixed with Se—CO (5 wt % to PS4Br) was prepared in tetrahydrofuran (THF) as the organic internal phase. Then 1 mL of this THF mixture solution was rapidly injected into the aqueous outer phase with vigorous stirring. The resulting suspension was subsequently sonicated for 10 min using a bath sonicator (Bransonic 2510-DTH) at a frequency of 42 kHz and power of 100 W. It was then further stirred at room temperature for 30 h to allow for the complete evaporation of organic solvents. Purification was done by washing the NPs in Milli-Q water 3 times using Amicon@ Ultra-4 centrifugal filters with a molecular weight cutoff of 30 kD. The purified SeCO—LPS4Br NPs were re-suspended in Milli-Q water to make 2.5 mg/mL stock concentration. The fluorescent dye co-encapsulated SeCO—IR780-LPS4Br NPs were synthesized using a similar procedure except when mixing PS4Br with organic phosphor Se—CO, IR-780 iodide (stock solution prepared in acetone at 1 mg/mL) was also added at 2 wt % to PS4Br.

To prepare SeCO—IR780-LPS4VP-PEG NPs for intravenous administration, PS4VP was used as the polymer matrix instead of PS4Br, and DSPE-PEG2000 (30 wt % to PS4VP) was used as the phospholipid instead of DMPA. To make 1 mL of the organic internal phase containing polymer, organic phosphor (5 wt % to polymer), and fluorescent dye (2 wt % to polymer), 400 μL THF was used to dissolve 10 mg of PS4VP, followed by adding 100 μL of Se—CO stock in THF at 5 mg/mL and 200 μL of IR-780 iodide stock in acetone at 1 mg/mL. Then 300 μL of acetone was added to the mixture to adjust the volume ratio of THF and acetone (1:1). The rest of the steps were similar to the nanoprecipitation procedure described above.

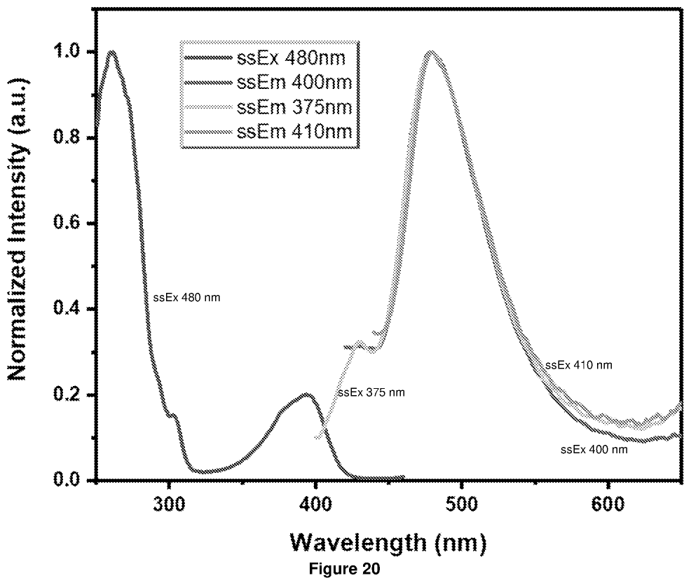

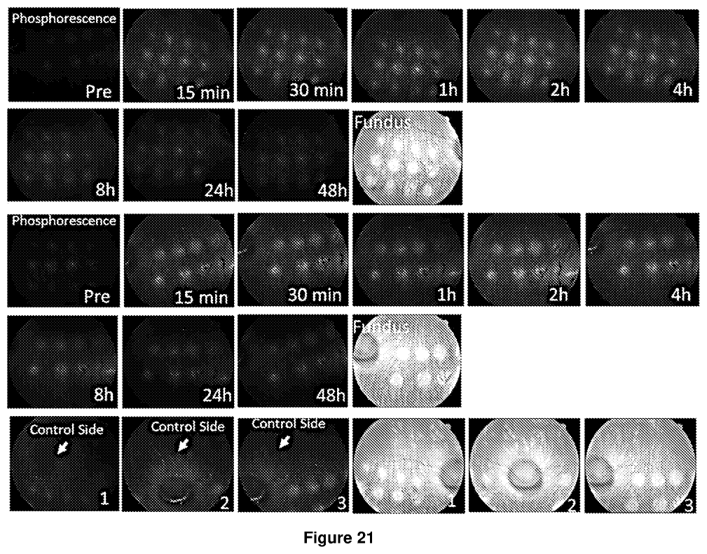

The SeCO—LPS4Br NPs exhibited average hydrodynamic size of 235.7 nm (PDI: 0.211), and the SeCO—IR780-LPS4VP-PEG NPs exhibited average hydrodynamic size of 63.6 nm (PDI: 0.183). In their water suspension, their emission were observed to be both very sensitive to oxygen. It was confirmed that the NPs could be excited with >400 nm light. FIG. 20 illustrates Steady state excitation (ssEx) and emission (ssEm) spectra of SeCO—LPS4Br NPs dispensed in water measured at 298K in anoxic condition. Emission curves were recorded with various excitation wavelengths

### Example 3—Animal Model Testing

All animal studies were implemented under the guidelines of the Association for Research in Vision and Ophthalmology (ARVO) Statement on the Use of Laboratory Animals in Ophthalmic and Vision Research. The experiment protocols were approved by the Institutional Animal Care and Use Committee (IACUC) of the University of Michigan (Protocol numbers: PRO00008566 and PRO00010388, PI: Y. Paulus).

A total of 11 rabbits were used in this study. Nine New Zealand white rabbits (2-4 months and 2.5-3.0 kg) were obtained from the Center for Advanced Models and Translational Sciences and Therapeutics (CAMTraST) at the University of Michigan Medical School, and two Dutch Belted rabbits (3 months and 1.3-1.5 kg) were purchased from Covance. The animals were divided into three groups: control, RVO, and CVO. In the RVO control group, the animals received intravitreal injection of organic RTP NPs (50 μL, 2.5 mg/mL) and had normal, healthy retinas. In the RVO group, the animals received hemi-retinal vein occlusion (RVO) by Rose Bengal dye-enhanced photochemical thrombosis laser photocoagulation. In the CVO group, the rabbits were treated with laser photocoagulation without administration of Rose Bengal. Throughout experiments and recovery, the animal condition including mucous membrane color, body temperature, heart rate, and respiratory rate was recorded and documented every 15 minutes. To induce anesthesia, a dose of ketamine (40 mg/kg) and xylazine (5 mg/kg) was injected intramuscularly (IM). The rabbit's pupils were dilated using tropicamide 1% ophthalmic and phenylephrine hydrochloride 2.5% ophthalmic. A drop of 0.5% topical tetracaine was applied for topical anesthesia before experiments. In addition, a drop of lubricant (Systane, Alcon Inc., TX, USA) was provided on the eye every minute to avoid corneal dehydration during the experiment. To maintain the animal's body temperature, a circulation heat blanket was used.

Retinal vein occlusion (RVO) model: Retinal vein occlusion (RVO) models were performed using the Rose Bengal dye-enhanced photochemical thrombosis as described in detail in previous studies including, Nguyen et al., Scientific reports 2019, 9 (1), 1-14; Nguyen et al., Biomed Opt Express 2018, 9 (12), 5915-5938; and Oncel et al., Retina (Philadelphia, Pa.) 1989, 9 (1), 1-7. Briefly, a 532 nm green light laser mounted on a slit lamp was used to create the RVO model (Vitra 532 nm, Quantel Medical, Cournon d'Auvergne, France). To visualize the target retina vessels, a contact lens (Volk H—R Wide Field, laser spot 2× magnification, Volk Optical Inc, Mentor, OH, USA) was placed on the cornea. When the target vessels were selected, rabbits under anesthesia were injected intravenously (IV) with Rose Bengal at a concentration of 50 mg/mL. 5-10 seconds after the injection, 20 spots of 532 nm laser at a power of 150 mW, aerial spot diameter of 75 μm, and pulse duration of 500 ms were illuminated into the retinal veins at the same position. The laser spots were applied at a distance of one-half to one disc diameter from the optic nerve to avoid side effects to the optic nerve such as optic neuropathy (Ho et al., Invest. Ophthalmol. Vis. Sci. 54, 5981-5988 (2013)). To avoid reperfusion of the vein, 20 laser spots were further applied with an increased power of 300 mW. The treated position on the veins was carefully selected to prevent damaging the adjacent arteries or optic nerve (Ameri et al., Ger J Ophthalmol 2008, 246 (10), 1429).

CVO model generation: To induce CVO model, a contact lens was placed on the cornea of the rabbit eye. Gonak Hypromellose Ophthalmic Demulcent Solution 2.5% was placed on the surface of the contact lens for coupling between the incident laser light and the cornea. Then, the rabbit eye was irradiated with a 532 nm green light laser at a power of 450 mW, aerial beam diameter of 150 μm, and pulse duration of 500 ms to create the CVO model using a Zeiss SL 130 slit lamp (Carl Zeiss Meditec, Jena, Germany), to which the Vitra photocoagulator was connected. Fifteen shots of the laser were illuminated into the eye at different positions. To create CVO model in Dutch Belted rabbits, the laser power was reduced from 450 mW to 300 mW due to strong absorption of laser light of melanin. Twelve spots were illuminated on the retina with aerial spot size of 150 μm and pulse duration of 500 ms. After taking color fundus photographs, fluorescein angiography (FA) and indocyanine green angiography (ICGA) were performed to evaluate the vasculature and confirm vascular occlusion.

Follow-up RVO and CVO evaluation: All rabbits with RVO model were examined fifteen minutes after the laser treatment, and at day 7 and 14 post-photocoagulation. The rabbit models were assessed by color fundus photography, fluorescein angiography (FA), indocyanine green (ICG) angiography, and phosphorescence photography.

Color Fundus: All retinal vessel network and laser-induced hypoxia were imaged using a custom-modified 50-degree color fundus photography (Topcon 50EX, Topcon Corporation, Tokyo, Japan). The digital images were captures by EOS 5D camera with a resolution of 5472×3648 pixels with a pixel size of 6.55 μm². Color fundus images were obtained using the maximum 50-degree angle of coverage centered at five different positions of the eye: the optic nerve, the superior retina above the optic disc, the inferior retina below the optic disc, the temporal medullary ray, and the nasal medullary ray. Color fundus montages were created using the 12K Retina software (Topcon Corporation, Tokyo, Japan).

Fluorescein angiography (FA) and indocyanine green angiography (ICGA): FA and ICGA were performed on the Topcon 50EX camera by changing the camera's appropriate internal excitation and emission filters for each. For FA, a dose of 0.2 mL fluorescein sodium at a concentration of 10% fluorescein (Akorn, Lake Forest, IL, USA) was intravenously injected into the rabbit via the marginal ear vein. For ICGA, 2.0 mL of ICG solution at concentration of 2.5 mg/mL (Akorn, Lake Forest, IL, USA) was injected intravenously via the marginal ear vein. FA and ICGA images were subsequently acquired after fluorophore injection, and late phase FA and ICGA images were acquired at every minute for a period of at least 20 minutes.

Phosphorescence photography examinations: To evaluate the organic RTP NPs as an oxygen sensor for detection of hypoxia in vivo, phosphorescence photography was evaluated on rabbit models using a custom-modified Topcon 50EX camera with custom-made filters (excitation filter with the bandpass wavelengths of 335 to 379 nm (FF01-357/44, Semrock, NY, USA) and barrier filter with a bandpass of 498 to 542 nm (FF01-520/44, FF01-357/44, Semrock, NY, USA) and UV-excitation light source with center wavelength of 365 nm and bandwidth of 9 nm (M365LP1, Thorlabs, USA). All rabbits with RVO model received intravitreal injection of 50 μL organic RTP NPs at a concentration of 2.5 mg/mL. Phosphorescent photography was acquired immediately after the injection and follow-ups for different time points: 15 min, 1, 2, 4, 8, 24 h and day 2, 4, and 7. For intravenous injection, the rabbits with CVO model were injected with 4 mL of RTP NPs at a concentration of 2.5 mg/mL. Phosphorescent imaging was acquired immediately after the injection and follow-ups for different time points: 15 min, 2, 4, 8, 16, 24 h, 48 h and day 7. The control groups were monitored over a period of 17 days post-injection.

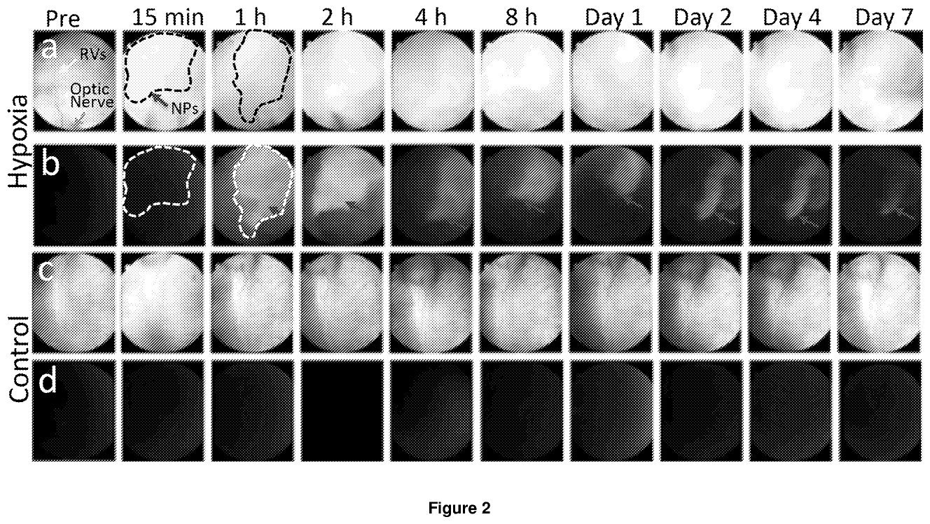

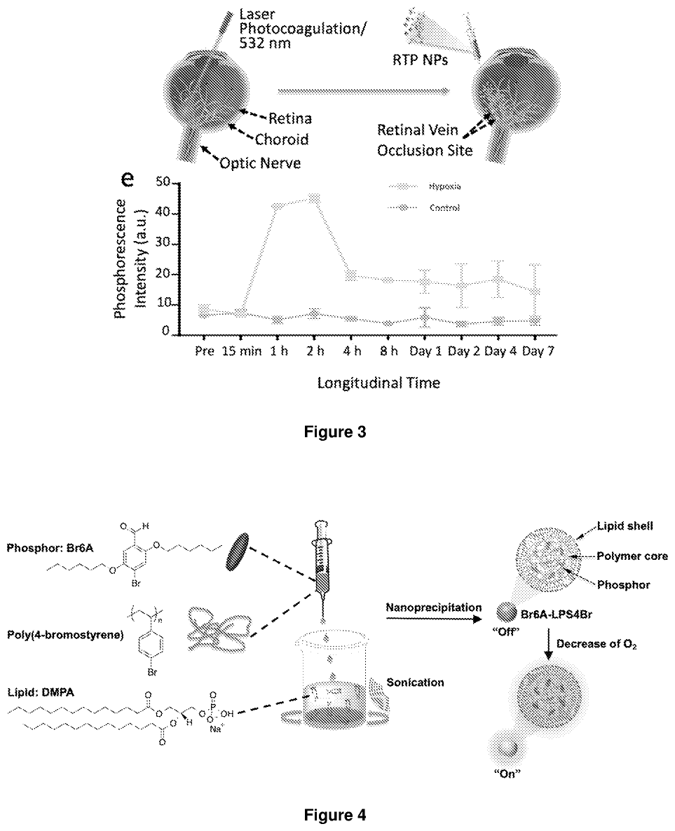

FIGS. 2 and 3 show longitudinal phosphorescence imaging of intravitreal Br6A-LPS4Br nanoparticles in living rabbit retinal hypoxia and control over 7 days (λₑₓ=365 nm, λₑₘ=530 nm). FIG. 2 (row a) and (row c) show color fundus photographs of the hypoxic (row a) and control normoxic side (row c) of the same rabbit before and after intravitreal administration of Br6A-LPS4Br NPs (50 μL, 2.5 mg/mL) at different time points (15 min, 1, 2, 4, 8 h and day 1, 2, 4, and 7). The color fundus photographs show rabbit fundus features such as retinal vessels (RVs), choroidal vessels, optic nerve, and distribution of nanoparticles in the vitreous. FIG. 2 (row b) and (row d) Phosphorescence images of the hypoxic (row b) and the control normoxic (row d) side before and up to 7 days after intravitreal administration of Br6A-LPS4Br NPs. Black dotted circles (row a) and white dotted circles (row b) indicate the position of nanoparticles. The arrows in row b indicate the location of phosphorescence signal. FIG. 3 shows average phosphorescence intensity measured from the hypoxic side and the control side. Error bars show the standard deviations of three independent measurements. The phosphorescence signal increased significantly on the hypoxic side by 1 h post-injection, peaked at 2 h post-injection, and persisted for at least 7 days.

To evaluate the efficiency of organic RTP NPs as in vivo nanosensors for the detection of tissue hypoxia, the synthesized Br6A-LPS4Br NPs were administrated intravitreally into six rabbits with localized hypoxia after Rose Bengal infusion and laser-induced hemi-RVO as described previously (Nguyen et al., Scientific reports 9, 1-14 (2019); Nguyen et al., Biomed Opt Express 9, 5915-5938 (2018)). In brief, the hypoxia model was obtained after illumination of one side of the retinal vessels with 532 nm laser light at a power of 150-300 mW, aerial spot diameter of 75 μm, and pulse duration of 500 ms on the rabbit retina to occlude the retinal vasculature. One-week post laser-induced RVO, the region of hypoxia was imaged with slit lamp examination, color fundus photography, and fluorescein angiography (FA). After acquiring baseline images, 50 μL of Br6A-LPS4Br NPs at a concentration of 2.5 mg/mL was administrated into the rabbits via intravitreal injection. Longitudinal distribution of the NPs was monitored for up to 7 days post-injection using color fundus photography and fundus phosphorescence imaging (FIG. 2). The phosphorescence intensity data was determined using the regions of interest (ROI) analysis method. FIG. 2 shows in vivo longitudinal visualization of hypoxia and the surrounding retinal vasculature pre- and post-administration of Br6A-LPS4Br NPs at different time points such as 15 min, 1, 2, 4, 8, and 24 hours, and 2, 4, and 7 days. FIG. 2 (row a and row c) illustrate the color fundus images of two different sides of the same rabbit eye: the hypoxic RVO side and the normoxic control (untreated) side. These fundus images depict retinal anatomy such as retinal vessels (arrows labeled “RV”) and the optic nerve (arrows). FIG. 2 (row b and row d) exhibit the corresponding phosphorescence images. There was no phosphorescent signal observed on the images before the injection of Br6A-LPS4Br NPs. Notably, the phosphorescent signal in the hypoxic region was not visible immediately after the injection (15 min post-injection) since the administrated Br6A-LPS4Br NPs suspension was initially saturated with air. It takes time for the dissolved oxygen to be depleted at the hypoxia site, and to subsequently turn on NPs' RTP signal. The NPs were clearly visualized starting at 1 h post-injection (FIG. 2 row b). These images demonstrate dynamic changes of the RTP signal of Br6A-LPS4Br NPs over time. The location of NPs is clearly visualized at 1-2 h post-injection and is still visible up to 7 days post-injection on the hypoxic side. This contrast indicates the ability of these organic RTP NPs to track tissue hypoxia in vivo. As expected for the untreated side, no evidence of phosphorescence contrast was detected over time given the normal oxygen tension (FIG. 2 row d). By applying an image segmentation algorithm to separate the contrast derived from the distribution of Br6A-LPS4Br NPs, average phosphorescence intensity (API) was quantified for each time point (FIG. 3). This quantification shows that API significantly increased post-injection compared to pre-injection of Br6A-LPS4Br NPs. The API in the hypoxia increased by 4.97-fold over the first hour post-injection from 8.58±1.46 (a.u.) pre-injection to 42.67±0.07 (a.u.) (p<0.001) and reached a peak at 2 h post-injection (API=45.13±1.31 (a.u.)), yielding a signal-to-noise ratio (“SNR”) of 12.5 using First Standard Deviation method). Although the API then decreased over time, it was still 168% higher at day 7 (API=14.45±8.86 (a.u.)) compared to pre-injection.

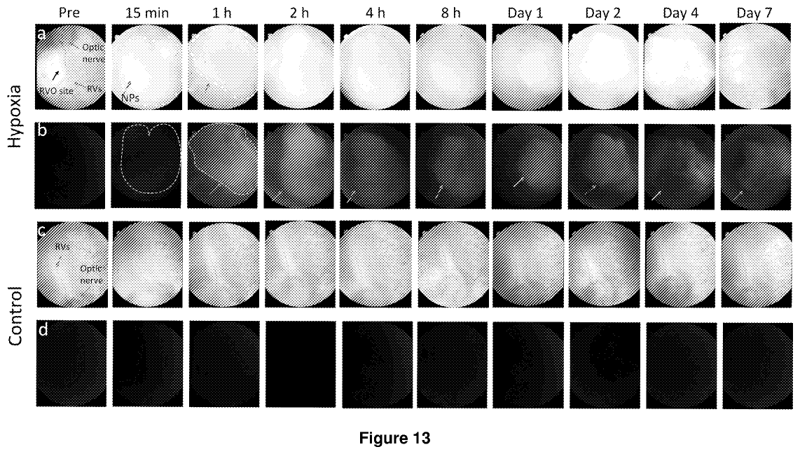

The in vivo reproducibility of retinal hypoxia detection by organic RTP NPs was verified in another rabbit, in which hypoxia was created via Rose Bengal infusion and laser-induced RVO two weeks prior to intravitreal injection of Br6A-LPS4Br NPs with the same dose and concentration (50 μL, 2.5 mg/mL) (FIG. 13). Hypoxia was monitored for 7 days. Phosphorescence images showed high contrast and peaked at 2 h post-injection, which is similar to the rabbit that received the injection 1 week after laser photocoagulation, reconfirming the in vivo hypoxia detecting and tracking capability of these organic RTP NPs.

FIG. 13 shows in vivo phosphorescence imaging of the retinal hypoxia acquired at different time points post-injection of Br6A-LPS4Br nanoparticles in a living rabbit. FIG. 13 (row a and c) shows color fundus photograph of the hypoxic (row a) and the control normoxic side (row c) acquired from the same rabbit eye pre- and post-injection of Br6A-LPS4Br nanoparticles (50 μL, 2.5 mg/mL) at day 14 post laser-induced RVO model. The arrows in row b and row a, 15 min and 1 h show the position of Br6A-LPS4Br nanoparticles after injection. FIG. 13 (row b and row d) shows in vivo phosphorescence images of the hypoxic (row b) and normal control side (row d) acquired at pre, 15 min, 1, 2, 4, 8 h and day 1, 2, 4, and 7 post-injection of Br6A-LPS4Br nanoparticles. The distribution of RTP nanoparticles post-injection is co-registered with the fundus color photograph (white dotted circle). RTP nanoparticles clearly showed up on phosphorescence images at 1 h post-injection with high contrast.

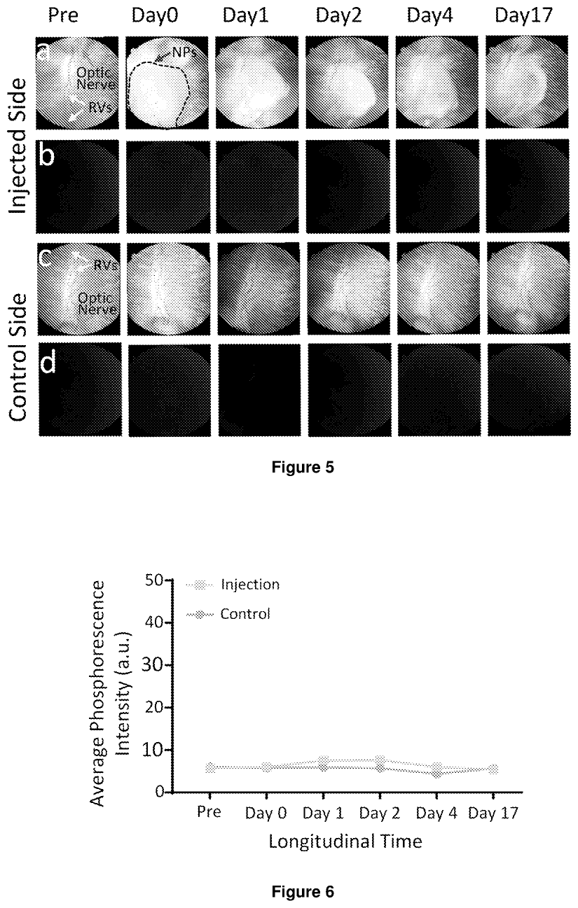

To further validate that the phosphorescence signal was activated by local hypoxia, Br6A-LPS4Br NPs (50 μL, 2.5 mg/mL) was injected into a normal, healthy rabbit and imaged with color fundus photography and fundus phosphorescence imaging at different time points over a period of 17 days (FIGS. 5 and 6). Phosphorescence signal was not detected either in the nanoparticle-injected side or in the non-injected side (FIG. 5 row b and row d) and ROI analysis shows that API did not change over time (FIG. 6). These results confirmed that the RTP signal of Br6A-LPS4Br NPs is quenched under normal oxygen tension in a healthy retina, and hence the developed organic RTP NPs can selectively detect ischemia-induced hypoxia.

FIGS. 5 and 6 show longitudinal phosphorescence imaging of a healthy control rabbit before and up to 17 days after the injection of Br6A-LPS4Br nanoparticles. FIG. 5 row a and row c show color fundus photography at the injection side (row a) and non-injection side (row c) in normal, healthy control rabbits. FIG. 5 row b and row d show corresponding phosphorescence images of the injection (row b) and non-injection (row d) side. Black dotted line in FIG. 5 (row a) shows the margin of RTP nanoparticles post-injection. FIG. 6 shows the average phosphorescence intensities from the injection and non-injection side over 17 days. No phosphorescence emission was observed on the phosphorescence images at any time point, since there is no tissue hypoxia.

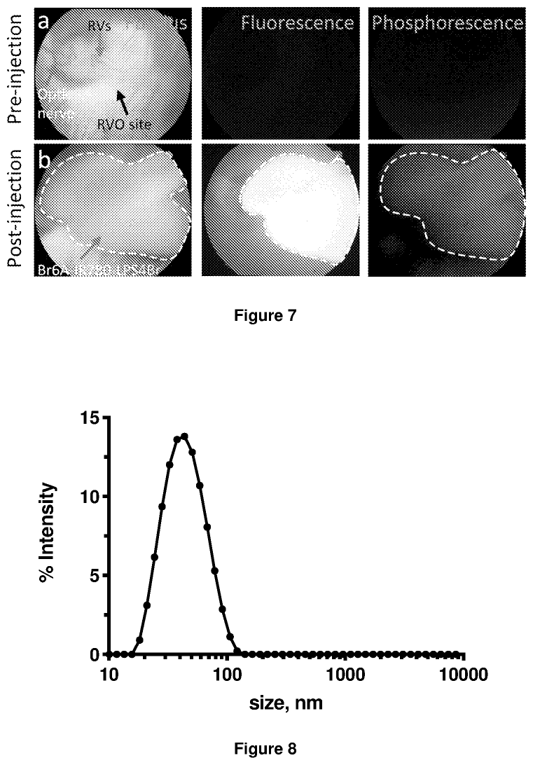

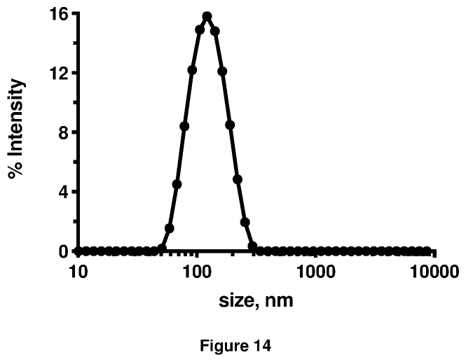

To confirm that the signal generated in the hypoxic area of the RVO model was truly arising from the phosphorescence emission of Br6A RTP NPs, a near-infrared fluorescent dye (IR-780) was co-encapsulated with the organic phosphor Br6A in the NPs, yielding narrow-dispersed (PDI: 0.108±0.012) Br6A-IR780-LPS4Br NPs with an average hydrodynamic diameter of 130.5±1.7 nm (FIG. 14), to allow dual phosphorescence and fluorescence imaging. Since the fluorescent signal of IR-780 is not affected by oxygen tension change, co-localizing the fluorescence and phosphorescence signals using different wavelength channels allows us to track the post-injection distribution of RTP NPs. Intravitreal injection of Br6A-IR780-LPS4Br NPs (50 μL, 2.5 mg/mL) into the RVO rabbit showed co-localization of the fluorescence signal of IR780 and phosphorescence signal (FIG. 7), indicating the hyperphosphorescence signal in the RVO model originates from Br6A within the RTP NPs.

In vivo multimodal fluorescence and phosphorescence images of intravitreal Br6A-IR780-LPS4Br NPs and retinal hypoxia in living rabbits: FIG. 7 shows fundus photography, fluorescence, and phosphorescence images obtained before (row a) and after (row b) intravitreal administration of 50 μL of Br6A-IR780-LPS4Br NPs at a concentration of 2.5 mg/mL. White dotted lines show the distribution of RTP NPs in the vitreous post-injection, the corresponding fluorescence emission from IR-780, and the corresponding phosphorescence emission from Br6A. The results show close correlation between the fluorescent and phosphorescent signal, indicating that the phosphorescent signal in the rabbit RVO model is emanating from RTP NPs.

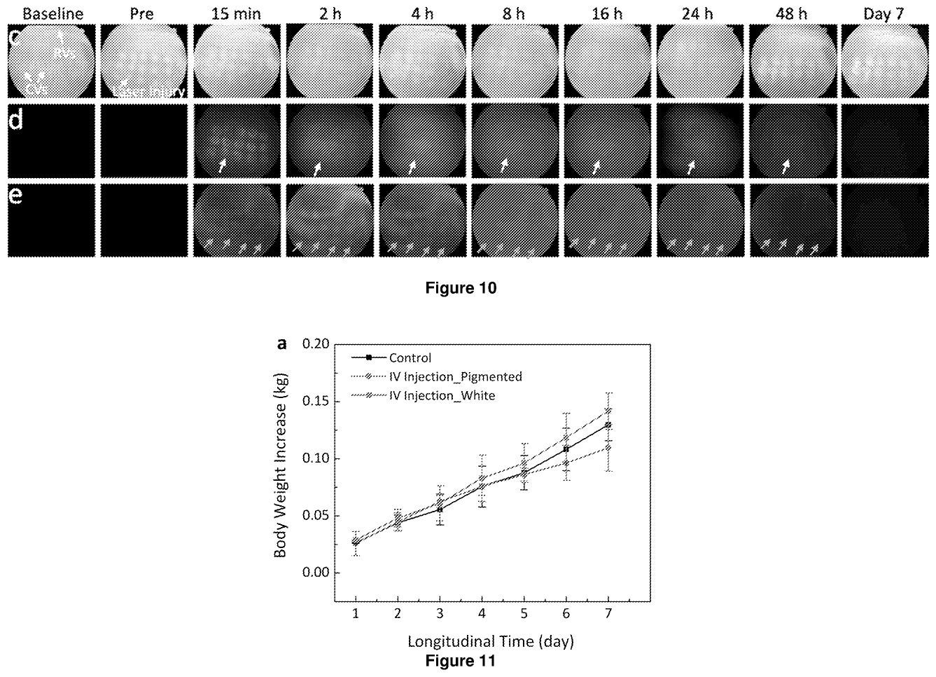

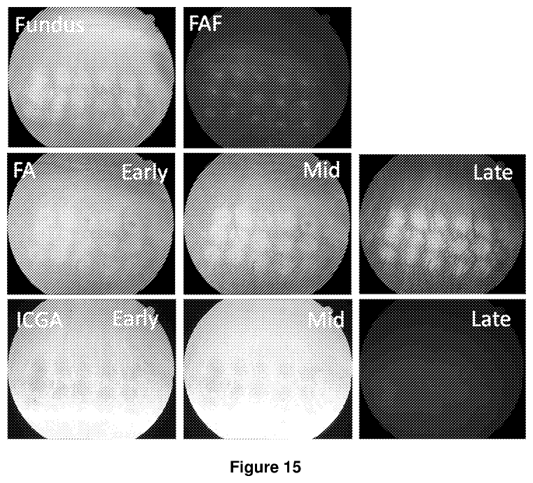

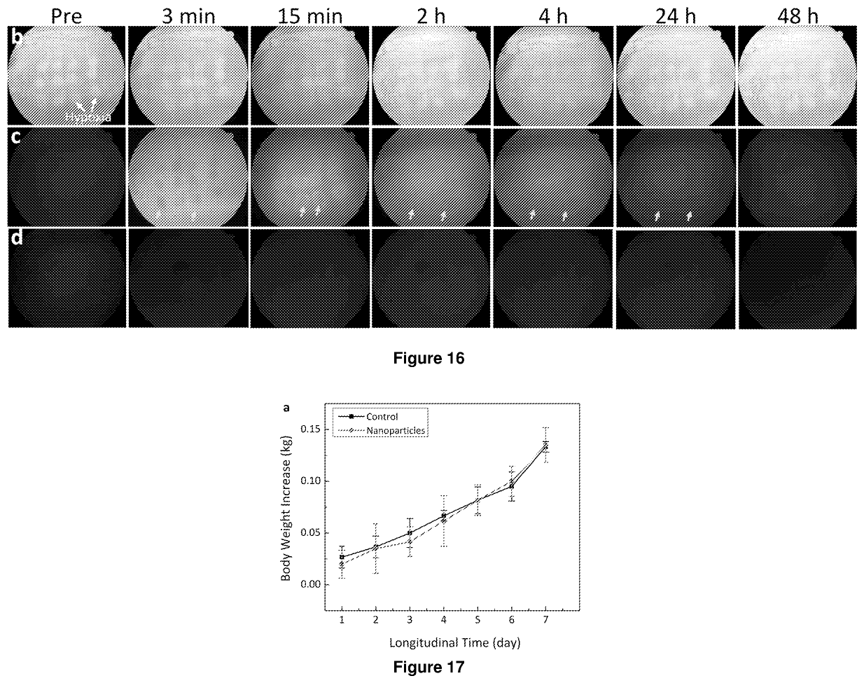

In vivo phosphorescence imaging of intravenous administered organic RTP NPs in rabbit CVO models: To realize minimally invasive delivery of these organic RTP NPs to retinal tissue for in vivo hypoxia imaging, an RTP NPs formulation suitable to be administrated intravenously was identified. Nanotherapeutics via intravenous delivery are usually formulated with average sizes of 10-150 nm in diameter to reduce reticuloendothelial system (RES) clearance and evade the 5 nm renal filtration cut off (Blanco et al., Nat. Biotechnol. 33, 941-951 (2015); Choi, H. S. et al., Nat. Biotechnol. 25, 1165-1170 (2007)). Unlike normal blood vessels with endothelial tight junctions and blood-retina barrier, occluded retinal veins tend to be disorganized and demonstrate hyperpermeability (Diaz-Coranguez, et al., Vision Res. 139, 123-137 (2017); Kusuhara et al., Diabetes Metab. J. 42, 364-376 (2018)). With these design criteria in mind, a hypothesis was tested, that smaller RTP NPs in the sub-100 nm range will more effectively extravasate and accumulate at occluded sites, leveraging the enhanced permeability and retention (EPR) effect. They will also likely to have higher diffusivity, allowing for more efficient penetration into the hypoxic chorioretinal tissue. Therefore, a more hydrophilic diblock copolymer, polystyrene-b-poly(4-vinylpyridine) (PS4VP, PS block, MW 35.5 kD and P4VP block MW 4.4 kD) was used, to construct NPs' polymeric core, which leads to smaller RTP NPs via bulk nanoprecipitation. IR-780 was co-encapsulated for particle location tracking in blood vessels and the retina. To improve RTP NPs' biocompatibility and pharmacokinetics for in vivo imaging via systemic delivery, a lipid-PEG conjugate, 1,2-distearoyl-sn-glycero-3-phosphoethanolamine-N-[carboxy(polyethylene glycol)-2000] (DSPE-PEG2000), was used instead of DMPA to create a “stealth” shell on the surface of the organic RTP NPs. Polyethylene glycol (PEG) is the most widely used “stealth” polymer in the drug delivery field, due to its long history of safety in humans and classification as Generally Regarded as Safe (GRAS) by the U.S. Food and Drug Administration (FDA) (Suk et al., Adv. Drug Deliv. Rev. 99, 28-51 (2016)). DSPE-PEG2000 has been approved for medical applications by the FDA. By means of a similar nanoprecipitation procedure as described before, narrow-dispersed (PDI: 0.134±0.005) RTP NPs Br6A-IR780-LPS4VP-(DSPE-PEG2000) with an average hydrodynamic diameter of 46.1±0.6 nm were successfully synthesized (FIG. 8). The choroid is a vascular-rich tissue immediately deep to the retina which supplies oxygen to the outer half of the retina including the fovea, or central vision, in humans, and thus it is important to understand both tissue oxygen tension within the retina and choroid. Using an established rabbit model of CVO with laser photocoagulation burns (FIG. 15), intravenously administrated Br6A-IR780-LPS4VP-(DSPE-PEG2000) NPs was found to be able to effectively extravasate through fenestrated blood vessels and accumulate at the laser lesions in the choroid and detect focal tissue hypoxia with a peak signal at 2 h post-injection (FIGS. 10 and 11). To confirm the phosphorescent signal detected was attributed to RTP NPs selectively sensing hypoxia rather than tissue autofluorescence, NPs encapsulated with IR-780 only and no Br6A (IR780-LPS4VP-(DSPE-PEG2000) NPs) was injected intravenously into rabbits with CVO. Consequently, only the fluorescent signal showing NPs location was observed, and no hyperphosphorescence at the occluded hypoxic laser lesions (FIG. 16).

To increase the clinical translatability, the in vivo reproducibility of chorioretinal hypoxia detection by Br6A-IR780-LPS4VP-(DSPE-PEG2000) NPs via IV injection was verified in multiple Dutch Belted pigmented rabbits (FIG. 16). These RTP NPs initially clustered at the rim of laser lesions (15 min) and gradually penetrated into the center region. At 4 h, patches of RTP NPs accumulated at center regions of laser lesions can be clearly visualized on the fluorescence channel (FIG. 16). Strong fluorescence signal was still detectable in both retinal and choroidal blood vessels even at 48 h post-injection, suggesting that Br6A-IR780-LPS4VP-(DSPE-PEG2000) NPs exhibit prolonged blood circulation.

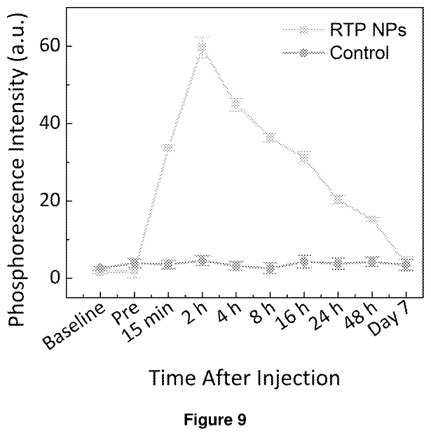

In vivo phosphorescence images of Br6A-IR780-LPS4VP-(DSPE-PEG2000) nanoparticles and chorioretinal hypoxia in living rabbits: FIG. 8 shows a dynamic light scattering measurement of Br6A-IR780-LPS4VP-(DSPE-PEG2000) NPs fabricated from PS4VP and DSPE-PEG2000. Narrow-dispersed (PDI: 0.134±0.005), fluorescent dye (IR-780) and organic phosphor (Br6A) co-encapsulated RTP nanoparticles with an average hydrodynamic diameter of 46.1±0.6 nm were synthesized using a similar nanoprecipitation method. FIG. 10 shows color fundus photography (row c), fluorescence (row d), and phosphorescence (row e) images before and after intravenous injection of 4 mL Br6A-IR780-LPS4VP-(DSPE-PEG2000) NPs at a concentration of 2.5 mg/mL. Color fundus images in FIG. 10 row c show the healthy retinal vessels (RVs), choroidal vessels (CVs) as well as the location of laser injured sites (white spots). Fluorescence and phosphorescence images obtained before and post injection at different time points, demonstrating the accumulation of RTP NPs at laser lesions (white arrow) and the corresponding phosphorescent signal from the RTP NPs (red arrows) detecting tissue hypoxia. FIG. 9 shows the quantification of mean phosphorescence intensities at laser lesions over 7 days. Phosphorescent signal was not visible before the injection of RTP NPs or on the normoxic control side. In contrast, the phosphorescent signal increased significantly in the hypoxic areas at 15 min post-injection, peaked at 2 h post-injection, and gradually decreased after that. Error bars show the standard deviations of three independent measurements.

Referring to FIGS. 20, SeCO—IR780-LPS4VP-PEG NPs were also tested in rabbits with the choroidal vascular occlusion (CVO) model described herein. Under 400 nm excitation with the Fundus camera, NPs accumulated at the laser-burn areas displayed bright emission which showed high contrast to the non-treated areas.

### Example 4—Biosafety Analysis

Biosafety analysis was performed on the treated animals using different methods such as body weight analysis, hematoxylin and eosin (H&E) staining, TUNEL assay, liver function test (LFTs), and kidney function tests (KFTs). Body weight was measured daily post administration of RTP NPs for each group over a period of 7 days. At day 14 after injection of RTP NPs, 400 μL of blood sample was collected from each rabbit for LFTs and KFTs. Then, the rabbits were euthanized by intravenous injection of euthanasia solution (0.22 mg/kg) via the marginal ear vein (Beuthanasia-D Special, Intervet Inc., Madison, NJ, USA). The organs and eye tissues were harvested and fixed with 10% neutral buffered formalin (VWR, Radnor, PA, USA). To prevent retinal detachment, eye tissues were fixed with Davidson's fixative solution (Electron Microscope Sciences, PA, USA) for 24 h. Afterwards, the samples were placed in 50% alcohol solution for 8 h and then replaced with 70% alcohol solution and kept at room temperature for 24 h. The fixed tissues were embedded in paraffin, sectioned into 6 μm thick sections, and stained with hematoxylin and eosin (H&E) for histopathological examination. TUNEL assay analysis were performed using TUNEL in situ Cell Death Detection Kit protocol (Sigma-Aldrich, USA). The stained slides were analyzed using DM6000 microscope. H&E images were captured using the BF450C camera and TUNEL fluorescence images were obtained using the FF363×camera (DM600, Leica Biosystems, Nussloch, Germany).

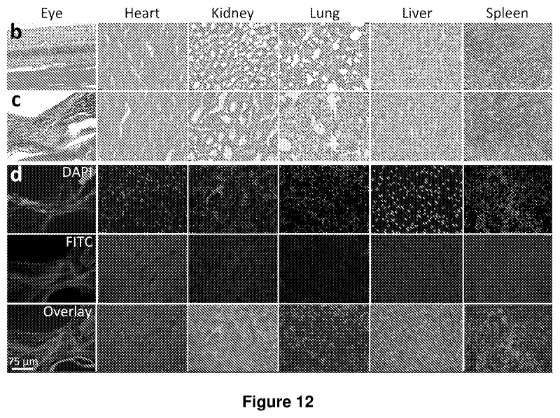

Biosafety evaluation in living rabbits: FIG. 11 shows a graph of the body weight increase measured daily for 7 days from three different groups: untreated control and intravenously treated CVO models in White New Zealand and Dutch Belted rabbits, showing no evidence of systematic toxicity through equivalent, appropriate weight gain in both RTP NPs treated and untreated control groups. FIG. 12 row b and row c shows H&E staining of tissues obtained 1-month post-intravenous injection of Br6A-IR780-LPS4VP-DSPE-PEG NPs (row b) compared to the untreated control group (row c), demonstrating preserved cellular morphology and nuclei without fragmentation or extracellular debris from dead cells in all of the evaluated organs, including eye, heart, kidney, lung, liver, and spleen. FIG. 12 (row d) shows a TUNEL assay analysis at 1-month post-intravenous injection of Br6A-IR780-LPS4VP-(DSPE-PEG2000) NPs. DAPI indicates cell nuclei. Green color stained with FITC evaluates for any apoptotic cells, which are not noted. Scale bar: 75 μm.

Table 1 below shows the results of the liver and kidney Function tests for untreated control, intravenously treated CVO model in New Zealand white and Dutch Belted rabbits, demonstrating normal levels at 14 days post-administration.

TABLE 1

| | | | Br6A-IR780-LPS4VP-PEG | | | |
|---|---|---|---|---|---|---|
| Nanoparticles Administrated | | | NZ | | | Control |
| Liver Function | Normal | | White | Pigmented | Pigmented | NZ White |
| Tests (LFT) | Range | Unit | Rabbit | Rabbit #1 | Rabbit #2 | Rabbit |
| Albumin | 2.7-5 | g/dL | 3.7 | 4.1 | 4.2 | 4.0 ± 0.3 |
| Total Protein | 5-7.5 | g/dL | 5.3 | 6 | 6.2 | 5.8 ± 0.5 |
| Alanine | 25-65 | U/L | 35 | 43 | 41 | 39.7 ± 4.2 |
| aminotransferase | | | | | | |
| (ALT) | | | | | | |
| Alkaline | 10.0-86.0 | U/L | 76 | 86 | 66 | 76.0 ± 10.0 |
| phosphatase | | | | | | |
| (ALP) | | | | | | |
| Total Bilirubin | 0.2-0.5 | mg/dL | 0.4 | 0.3 | 0.2 | 0.3 ± 0.1 |
| (TBIL) | | | | | | |
| Kidney Function Tests (KFT) | | | | | | |
| Blood urea | 5.0-25.0 | mg/dL | 15 | 21 | 21 | 19 ± 3.5 |
| nitrogen (BUN) | | | | | | |
| Creatinine (CREA) | 0.5-2.6 | mg/dL | 0.67 | 0.68 | 1.11 | 0.8 ± 0.2 |
| Calcium | 5.6-12.1 | mg/dL | 12.0 | 14 | 12.9 | 13.0 ± 1.0 |
| Glucose | 74-148 | mg/dL | 137 | 111 | 189 | 145.7 ± 39.7 |

TABLE 2

| Liver Function Tests | Normal | | | Treated | Treated | Treated |
|---|---|---|---|---|---|---|
| (LFT) | Range | Unit | Control | #1 | #2 | #3 |
| Albumin | 2.7-5 | g/dL | 4.2 | 3.4 | 3.5 | 3 |
| Total Protein (TPRO) | 5-7.5 | g/dL | 6.6 | 6.7 | 5 | 7.3 |
| Alanine | 25-65 | U/L | 31 | 25 | 51 | 29 |
| aminotransferase | | | | | | |
| (ALT) | | | | | | |
| Alkaline phosphatase | 10.0-86.0 | U/L | 77 | 81 | 51 | 85 |
| (ALP) | | | | | | |
| Total Bilirubin (TBIL) | 0.2-0.5 | mg/dL | 0.4 | 0.3 | 0.33 | 0.22 |

Kidney Function Tests (KFT)

| | | | | | | |
|---|---|---|---|---|---|---|
| Blood urea nitrogen | 5.0-25.0 | mg/dL | 25 | 20 | 15 | 17 |
| (BUN) | | | | | | |
| Creatinine (CREA) | 0.5-2.6 | mg/dL | 1.35 | 0.68 | 0.78 | 0.74 |
| Calcium | 5.6-12.1 | mg/dL | 11.2 | 11.4 | 11.7 | 9.7 |
| Glucose | 74-148 | mg/dL | 102 | 122 | 133 | 114 |

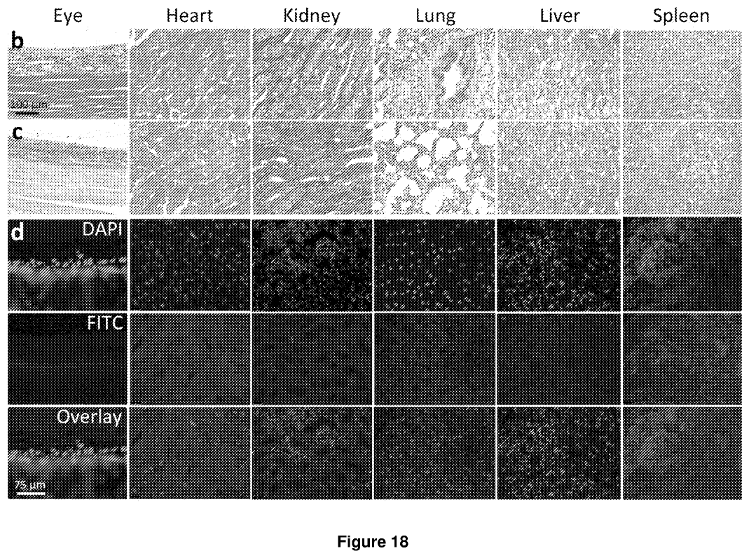

To test the possible toxicity of IV and intravitreal administration of RTP NPs in these rabbits, several biosafety analyses were performed, including body weight analysis, liver function tests (LFT), kidney function tests (KFT), TUNEL assay, and histopathology. All rabbits used in this study had their weight measured daily with and without the injection of Br6A-LPS4Br NPs (FIG. 11 and FIG. 17). Body weight of intravenously treated, intravitreally treated, and untreated groups increased appropriately during the 7 days after each treatment condition, demonstrating that these RTP NPs do not induce negative systemic impact on living rabbits. Hematoxylin and eosin (H&E) staining demonstrated preserved normal retinal cellular morphology and nuclei without fragmentation or extracellular debris from dead cells (FIG. 12 row b and row c, and FIG. 18 row b and row c) TUNEL assay analysis demonstrates no evidence of cells undergoing apoptosis in vivo in the eye (FIG. 12 (row d) and FIG. 18 (row d)). Serum blood tests were performed to examine the acute toxicity of the RTP NPs to liver or kidney function of the animals. As shown in Table 1 for intravenous injection and Table 2 for intravitreal injection, alanine aminotransferase (ALT), alkaline phosphatase (ALP), creatinine (CRE), blood urea nitrogen (BUN), and other liver and kidney function tests are within normal range in all animals, indicating that the liver and kidney function of the rabbits were normal without adverse systemic toxicity for 14 days within administration.

Biosafety evaluation in living rabbits: FIG. 11 shows Body weight increase measured daily for 7 days from three different groups: untreated control and intravenously treated CVO models in White New Zealand and Dutch Belted rabbits, showing no evidence of systematic toxicity through equivalent, appropriate weight gain in both RTP NPs treated and untreated control groups. FIG. 12 row b and row c show H&E staining of tissues obtained 1-month post-intravenous injection of Br6A-IR780-LPS4VP-(DSPE-PEG2000) NPs (row b) compared to the untreated control group (row c), demonstrating preserved cellular morphology and nuclei without fragmentation or extracellular debris from dead cells in all of the evaluated organs, including eye, heart, kidney, lung, liver, and spleen. FIG. 12 (row d) shows TUNEL assay analysis at 1-month post-intravenous injection of Br6A-IR780-LPS4VP-(DSPE-PEG2000) NPs. DAPI indicates cell nuclei. Green color stained with FITC evaluates for any apoptotic cells, which are not noted. Scale bar: 75 μm.

Biosafety evaluation in living rabbits after intravitreal injection: FIG. 17 shows the body weight increase of untreated control and RTP NPs treated RVO model in White New Zealand rabbits via intravitreal injection measured daily for 7 days. The results showed that rabbit weights were gradually increased at similar rates for both treated and untreated groups, indicating no evidence of systematic toxicity caused by RTP NPs via intravitreal injection. FIG. 18 row b and row c show H&E images of tissues after intravitreal injection of Br6A-LPS4Br NPs (row b) compared to the untreated control group (row c). Scale bar: 100 μm. FIG. 18 (row d) shows TUNEL assay analysis. DAPI indicates cell nuclei. Green color stained with FITC evaluates for any apoptotic cells, which are not noted. Scale bar: 75 μm.

Room-temperature phosphorescence nanoparticles were successfully developed to generate efficient metal-free organic RTP NPs for real-time optical visualization of retinal hypoxia in living rabbits. The facile nanoformulation involves (1) a rigid and oxygen-permeable polymer matrix core for the effective activation of bright RTP from the embedded organic phosphor (Br6A) in hypoxic environments, and (2) an amphiphilic phospholipid shell, allowing for excellent water dispersity, biocompatibility, and colloidal stability. The phosphorescent signal of the fabricated organic RTP NPs demonstrates milliseconds decay time and is highly responsive toward oxygen quenching, which enables them to be exploited as “turn-on” imaging probes for retinal tissue hypoxia.

In vivo testing using rabbit RVO and CVO models, both oxygen-sensing efficacy and biosafety of the organic RTP NPs were demonstrated. The phosphorescence signal was exclusively generated in the areas of RVO or CVO where tissue hypoxia was present. No hyperphosphorescence was detected in healthy rabbits. The fluorescent dye and organic phosphor co-encapsulated NPs showed co-localization of fluorescence and phosphorescence signals, confirming the hyperphosphorescence in the RVO model originates from organic RTP NPs in response to tissue hypoxia. Since the light-generating mechanism of the organic phosphor Br6A in response to oxygen tension is at molecular scale, and RTP particles are at nanoscale, in theory it is reasonable to anticipate high spatial resolution (nanoscale) for in vivo hypoxia imaging, though in practice resolution will be more restricted to likely around 5 to 20 μm due to limited focusing of the light in the eye without adaptive optics. No ocular or systemic complications such as infection or toxicity were noted after either intravenous or intravitreal administration of RTP NPs. These data provide proof-of-concept that the developed nanoformulation of organic RTP materials allows for biocompatible, non-destructive detection of hypoxia in retinal tissue, currently unachievable by other methods.

This disclosure describes an imaging tool for monitoring tissue-level oxygen tensions in a non-destructive fashion. The results of this work provide a strong indication that the developed cost-effective organic RTP NPs hold significant promise as an advanced non-invasive imaging tool for monitoring retinal tissue oxygen levels and to evaluate retinal vascular diseases such as RVO. This has both scientific and translational impact at many levels to advance a number of disciplines and diseases in which tissue oxygen tension plays an important role in the pathophysiology. These organic RTP NPs can allow for improved understanding of the pathophysiology of vascular diseases by visualizing tissue ischemia with high spatiotemporal resolution. In addition, these organic RTP NPs can improve early diagnosis and prognosis research to enable real-time, in vivo oxygen tension determination and biomarker evaluation. While further studies will be conducted to assess this RTP nanosensor's hypoxia detection window, in vitro oxygen sensitivity calibration profile, and in vivo oxygen tension determination in the retina and choroid, this study lays a solid foundation demonstrating that this organic RTP nanosensor can serve as a general tissue hypoxia tracking probe via minimally invasive systemic delivery. This will enable improved understanding of the pathophysiology, and early diagnosis and prognosis research of diseases involving ischemia-induced hypoxia, beyond the retina and choroid.

Many modifications and other embodiments disclosed herein will come to mind to one skilled in the art to which the disclosed compositions and methods pertain having the benefit of the teachings presented in the foregoing descriptions. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

1. A room-temperature phosphorescence nanoparticle comprising:
a solid core comprising a metal-free organic phosphor embedded in a polymer, wherein the polymer is hydrophobic, glassy, and oxygen-permeable and the metal-free organic phosphor is present in an amount of about 0.1 wt % to about 20 wt %, based on the total weight of the polymer; and
a shell surrounding the core, the shell comprising a lipid.

2. The nanoparticle of claim 1, wherein the metal-free organic phosphor is one or more of 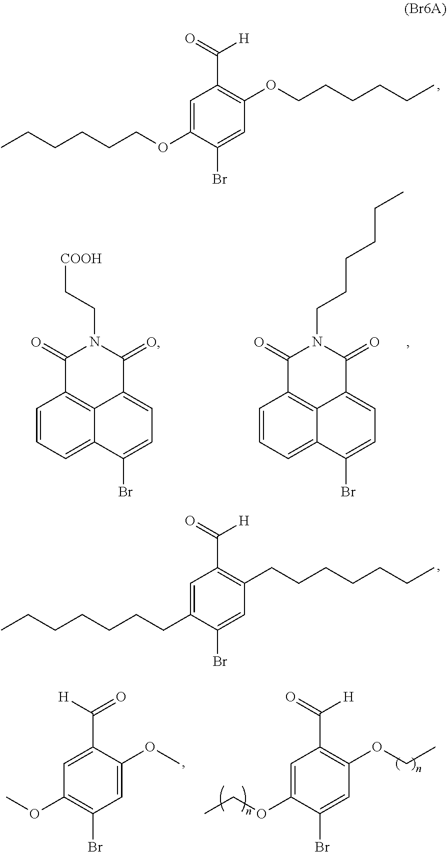
wherein n can be 4, 6, or 7, 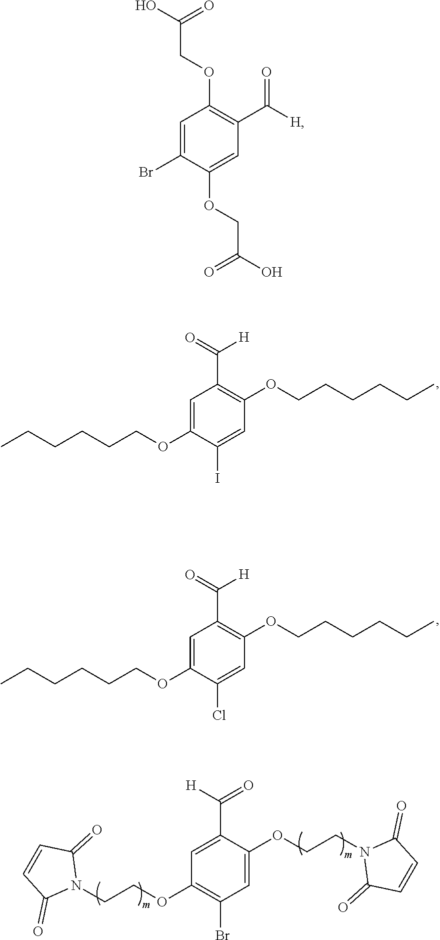
wherein m can be 1, 2, or 3,

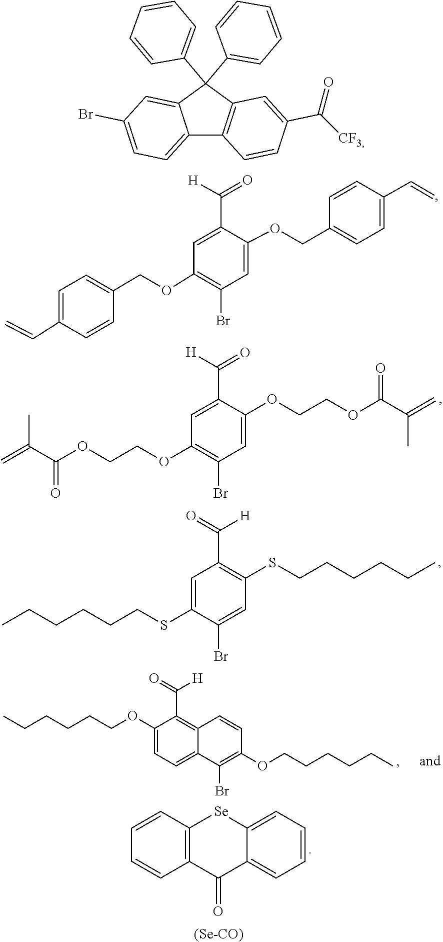

3. (canceled)

4. (canceled)

5. The nanoparticle of claim 1, wherein the polymer comprises a polystyrene homopolymer, a halogenated polystyrene, a polystyrene copolymer, or a combination thereof.

6. The nanoparticle of claim 5, wherein the polystyrene copolymer is polystyrene-b-poly(4-vinylpyridine) or wherein the halogenated polystyrene is poly (4-bromostyrene).

7. (canceled)

8. (canceled)

9. (canceled)

10. (canceled)

11. The nanoparticle of claim 1, wherein the lipid is amphiphilic and is one or more of 1,2-dimyristoyl-sn-glycero-3-phosphate (DMPA), 1,2-distearoyl-sn-glycero-3-phosphoethanolamine-N-[carboxy(polyethylene glycol)-2000](DSPE-PEG2000), a salt of 1,2-distearoyl-sn-glycero-3-phosphoethanolamine-N-[carboxy(polyethylene glycol)-2000] (DSPE-PEG2000), 1,2-distearoyl-sn-glycero-3-phosphoethanolamine-N-[methoxy(polyethylene glycol)-2000], a salt of 1,2-distearoyl-sn-glycero-3-phosphoethanolamine-N-[methoxy(polyethylene glycol)-2000], ammonium salt of 1,2-distearoyl-sn-glycero-3-phosphoethanolamine-N-[methoxy(polyethylene glycol)-2000], and polyethylene glycol.

12. The nanoparticle of claim 1 further comprising a fluorescent dye embedded in the polymer.

13. (canceled)

14. The nanoparticle of claim 1, wherein the metal-free organic phosphor is present in an amount of about 1 wt % to about 10 wt %, based on the total weight of the polymer.

15. (canceled)

16. A method of detecting hypoxia in a mammalian subject comprising administering to the subject the nanoparticle of claim 1, and obtaining phosphorescence images of the mammalian subject.

17. The method of claim 16, further comprising obtaining color fundus photography and fluorescence images of the mammalian subject.

18. The method of claim 16, wherein the nanoparticles are administered via intravenous injection, topical administration, or local injection.

19. (canceled)

20. (canceled)

21. The method of claim 20, wherein the nanoparticles are present in a suspension comprising and the nanoparticles are present in the suspension at a concentration in a range of about 0.01 mg/mL to about 25 mg/mL.

22. (canceled)

23. (canceled)

24. (canceled)

25. The method of claim 16, wherein the mammalian subject has chorioretinal hypoxia.

26. A method of preparing a room-temperature phosphorescence nanoparticle, comprising: adding a first solution comprising a first solvent, a polymer, and a metal-free organic phosphor to a stirring second solution comprising a second solvent and a lipid under conditions to form the room-temperature phosphorescence nanoparticle comprising a solid core comprising the metal-free organic phosphor embedded in a polymer and a shell surrounding the core comprising the lipid, wherein the polymer is hydrophobic, glassy, and oxygen-permeable, and the metal-free organic phosphor is present in an amount of about 0.1 wt % to about 20 wt %, based on the total weight of the polymer.

27. The method of claim 26, wherein the first solvent comprises an aprotic organic solvent and/or the second solvent comprises a protic organic solvent, water or both.

28. The method of claim 27, wherein the first solvent is one or more of tetrahydrofuran, acetonitrile, ethyl acetate, dimethylformamide (DMF), acetone, 1,4-dioxane, chlorobenzene, nitromethane, diethyl ether, triethyl amine, and dimethyl sulfoxide (DMSO); and/or
the second solvent comprises a protic organic solvent comprising one or more of methanol, ethanol, isopropyl alcohol, 1-butanol, 2-butanol, t-butyl alcohol, and acetic acid or the second solvent comprises ethanol and water.

29. The method of claim 28, wherein the first solution comprises the polymer in an amount of about 0.1 mg/mL to about 50 mg/mL.

30. (canceled)

31. The method of claim 26, wherein the first solution comprises the metal-free organic phosphor in an amount of about 0.1 wt % to about 10 wt % based on the total amount of polymer.

32. (canceled)

33. (canceled)

34. (canceled)

35. (canceled)

36. The method of claim 26, wherein the second solution comprises the lipid in an amount in a range of about 0.001 mg/mL to about 3 mg/mL.

37. (canceled)

38. The method of claim 26, wherein after the first and second solutions are stirred together, the first and second solutions are sonicated for about 1 minute to about 1 hour.

39. (canceled)

40. The method of claim 26, wherein the metal-free organic phosphor comprises one or more of 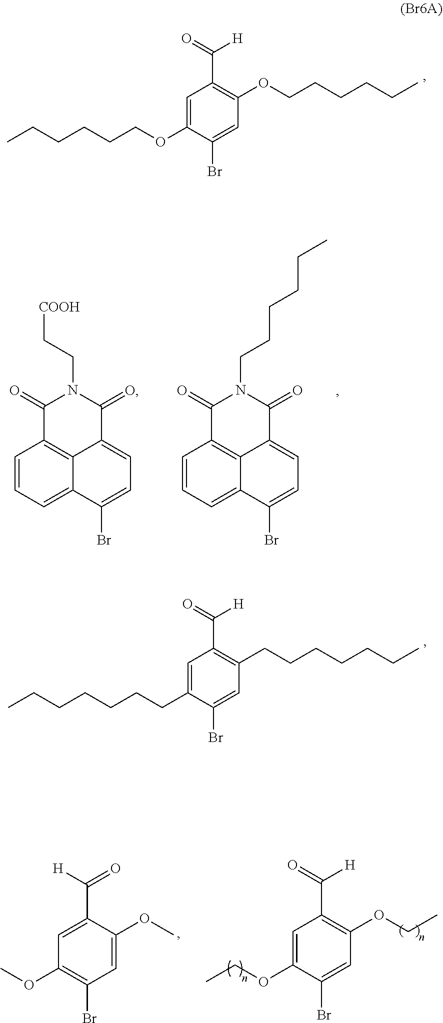
wherein n can be 4, 6, or 7, 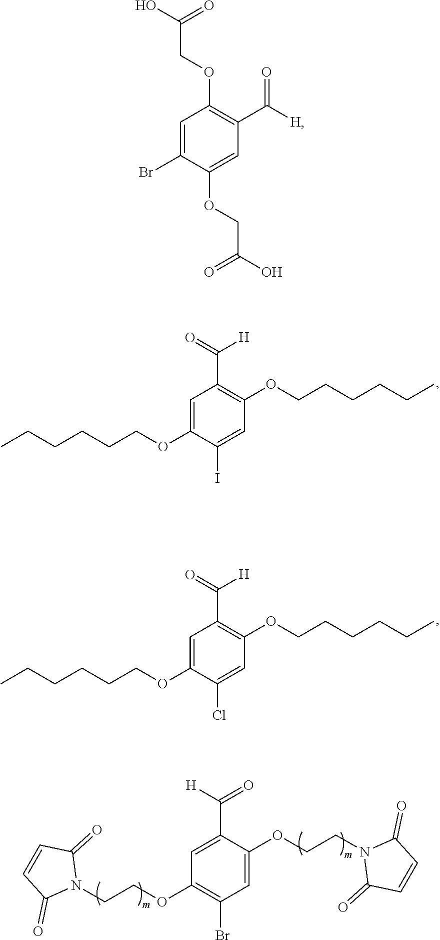
wherein m can be 1, 2, or 3,

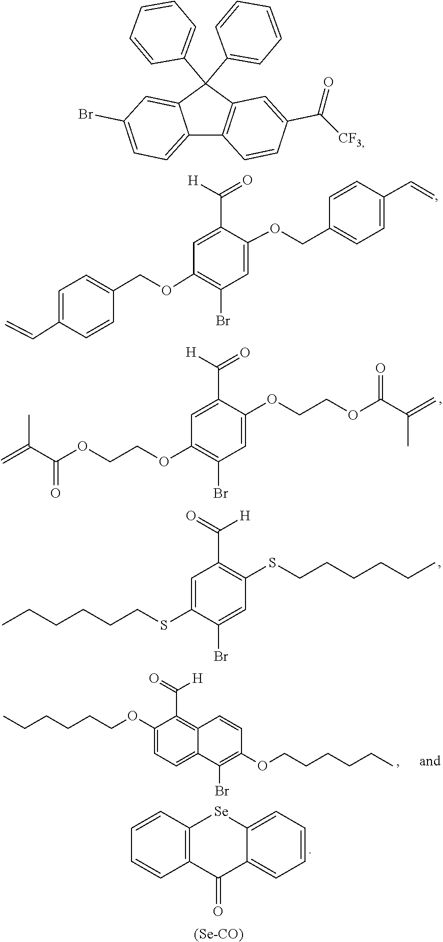

41. (canceled)

42. (canceled)
